(19) United States
(12) Patent Application Publication
(10) Pub. No.: US 2022/0293241 A1
(43) Pub. Date: Sep. 15, 2022

(54) SYSTEMS AND METHODS FOR SIGNALING COGNITIVE-STATE TRANSITIONS

(71) Applicant: Facebook Technologies, LLC, Menlo Park, CA (US)

(72) Inventors: Tanya Renee Jonker, Seattle, WA (US); Candace Peacock, Boulder, CO (US); Ting Zhang, Lake Jackson, TX (US); Brendan Matthew David-John, Gainesville, FL (US); Matthew Jordan Boring, Pittsburgh, PA (US); Thomas Scott Murdison, Seattle, WA (US); Yan Xu, Kirkland, WA (US); Hrvoje Benko, Seattle, WA (US)

(21) Appl. No.: 17/669,171

(22) Filed: Feb. 10, 2022

Related U.S. Application Data
(60) Provisional application No. 63/160,443, filed on Mar. 12, 2021.

(51) Int. Cl. G16H 20/70; A61B 5/16

(52) U.S. Cl. CPC G16H 20/70; A61B 5/165; A61B 5/163

(57) ABSTRACT

The disclosed computer-implemented method may include (1) acquiring, via one or more biosensors, one or more biosignals generated by a user of a computing system, (2) using the one or more biosignals to anticipate a transition to or from a cognitive state of the user, and (3) providing a signal indicating the transition to or from the cognitive state of the user to an intelligent-facilitation subsystem adapted to perform one or more assistive actions to reduce the user's cognitive load. Various other methods, systems, and computer-readable media are also disclosed.

### CROSS REFERENCE TO RELATED APPLICATION

This application claims the benefit of U.S. Provisional Application No. 63/160,443, filed 12 Mar. 2021, the disclosure of which is incorporated, in its entirety, by this reference.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 is a block diagram of an exemplary system for signaling and/or reacting to transitions to, from, and/or between the cognitive states of users, according to at least one embodiment of the present disclosure.

FIG. 2 is a diagram of exemplary cognitive states and corresponding transitions, according to at least one embodiment of the present disclosure.

FIG. 3 is a diagram of an exemplary data flow associated with an exemplary intelligent-facilitation subsystem, according to at least one embodiment of the present disclosure.

FIG. 4 is a block diagram of an exemplary wearable device that signals and/or reacts to cognitive-state transitions, according to at least one embodiment of the present disclosure.

FIG. 5 is a flow diagram of an exemplary method for signaling cognitive-state transitions, according to at least one embodiment of the present disclosure.

FIG. 6 is a diagram of an exemplary data flow for using biosensor data to generate signals of cognitive-state transitions, according to at least one embodiment of the present disclosure.

FIG. 7 is a diagram of an exemplary pre-processing data flow for generating gaze events and other gaze features from eye-tracking data, according to at least one embodiment of the present disclosure.

FIG. 8 is a flow diagram of an exemplary method for intelligently facilitating users' cognitive tasks and/or goals in response to cognitive-state transitions, according to at least one embodiment of the present disclosure.

FIG. 9 is a flow diagram of exemplary sub-steps for performing assistive actions to reduce cognitive loads associated with users' cognitive tasks and/or goals, according to at least one embodiment of the present disclosure.

FIG. 10 is a flow diagram of additional exemplary sub-steps for performing assistive actions to reduce cognitive loads associated with users' cognitive tasks and/or goals, according to at least one embodiment of the present disclosure.

FIG. 11 is an illustration of exemplary augmented-reality glasses that may be used in connection with embodiments of this disclosure.

FIG. 12 is an illustration of an exemplary virtual-reality headset that may be used in connection with embodiments of this disclosure.

FIG. 13 is an illustration of exemplary haptic devices that may be used in connection with embodiments of this disclosure.

FIG. 14 is an illustration of an exemplary virtual-reality environment according to embodiments of this disclosure.

FIG. 15 is an illustration of an exemplary augmented-reality environment according to embodiments of this disclosure.

FIG. 16 an illustration of an exemplary system that incorporates an eye-tracking subsystem capable of tracking a user's eye(s).

FIG. 17 is a more detailed illustration of various aspects of the eye-tracking subsystem illustrated in FIG. 16.

FIGS. 18A and 18B are illustrations of an exemplary human-machine interface configured to be worn around a user's lower arm or wrist.

FIGS. 19A and 19B are illustrations of an exemplary schematic diagram with internal components of a wearable system.

FIG. 20 is a schematic diagram of components of an exemplary biosignal sensing system in accordance with some embodiments of the technology described herein.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

Augmented Reality (AR) systems, Virtual Reality (VR) systems, and Mixed Reality (MR) systems, collectively referred to as Extended Reality (XR) systems, are a budding segment of today's personal computing systems. XR systems, especially wearable XR systems such as head-mounted XR systems, may be poised to usher in an entirely new era of personal computing by providing users with persistent “always-on” assistance, which may be integrated seamlessly into the users' day-to-day lives without being disruptive. In contrast to more traditional personal computing devices, such as laptops or smartphones, XR devices may be capable of displaying outputs to users in a more accessible, lower-friction manner. For example, some head-mounted XR devices may include displays that are always in users fields of view with which the XR devices may present visual outputs to the users.

Unfortunately, traditional XR devices often rely on input modalities (e.g., hand gestures or speech) that are cumbersome, ambiguous, lower precision, and/or noisier, which may make the information or tools provided by traditional XR devices difficult to access and navigate as well as physically and cognitively fatiguing. Some traditional head-mounted XR devices may attempt to automatically couple displayed outputs to users' physical environments (e.g., by placing labels or menus on real-world objects) such that users may more easily consume the displayed outputs. While easier to access, information displayed in this way may be distracting or annoying to users. Additionally, traditional XR devices often have interaction environments that are unknown, less known, or not prespecified, which may cause some XR systems to consume considerable amounts of computing resources to discover objects within their environments with which the XR devices may attempt to facilitate user interactions. If users have no immediate or future intentions to interact with the objects in their environments, any resources consumed in discovering the objects and/or possible user interactions may be wasted.

The present disclosure is generally directed to systems and methods for using biosignals (e.g., eye-tracking data or other biosignals indicative of gaze dynamics) to anticipate and signal, in real time, transitions to, from, and/or between a user's cognitive states, such as visual search, information encoding, rehearsal, storage, and/or retrieval. In some embodiments, the disclosed systems may anticipate when a user intends to encode information to the user's working memory and may intelligently perform (e.g., via adaptive and/or predictive interfaces) one or more assistive actions or interventions to reduce the physical and cognitive burdens involved in remembering and/or recalling the information. By anticipating the timing of a user's cognitive-state transitions, the systems and methods disclosed herein may responsively drive ultra-low-friction predictive interfaces to facilitate the user's cognitive tasks and goals. In some embodiments, the disclosed systems and methods may generate signals indicating the timing of a user's cognitive-state transitions that may allow intelligent facilitation systems to provide adaptive interventions at just the right time.

Features from any of the embodiments described herein may be used in combination with one another in accordance with the general principles described herein. These and other embodiments, features, and advantages will be more fully understood upon reading the following detailed description in conjunction with the accompanying drawings and claims.

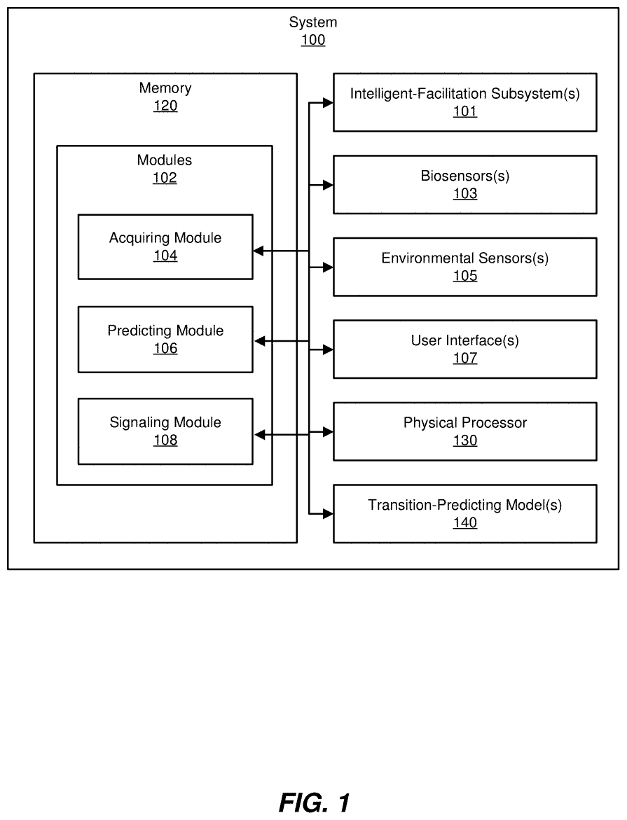

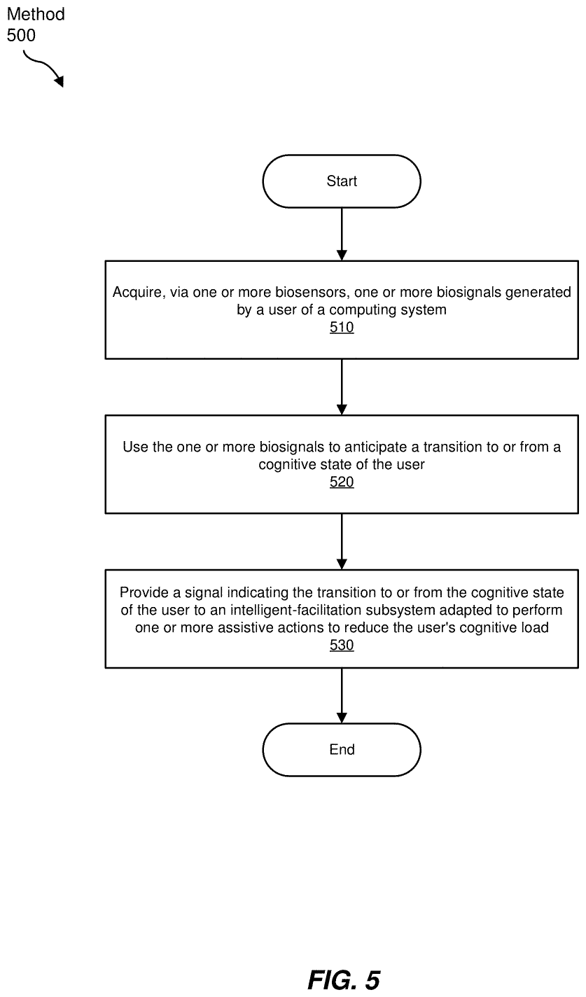

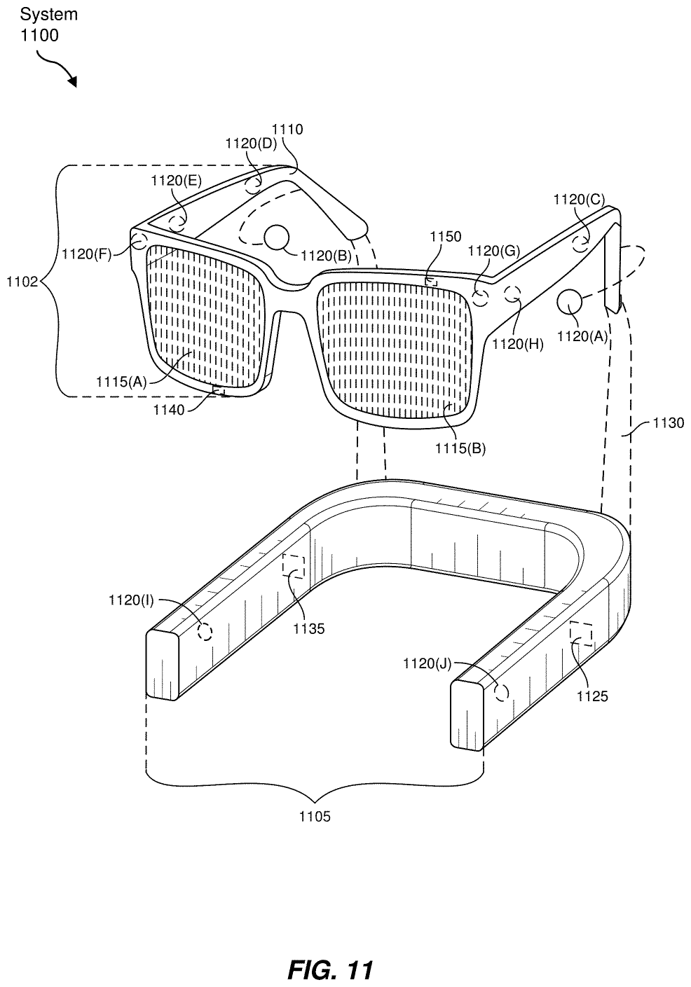

The following will provide, with reference to FIGS. 1-4, detailed descriptions of exemplary systems and subsystems for anticipating, signaling, and/or adapting to cognitive-state transitions. The discussions corresponding to FIGS. 5-10 will provide detailed descriptions of corresponding methods and data flows. Finally, with reference to FIGS. 11-20, the following will provide detailed descriptions of various extended-reality systems and components that may implement embodiments of the present disclosure.

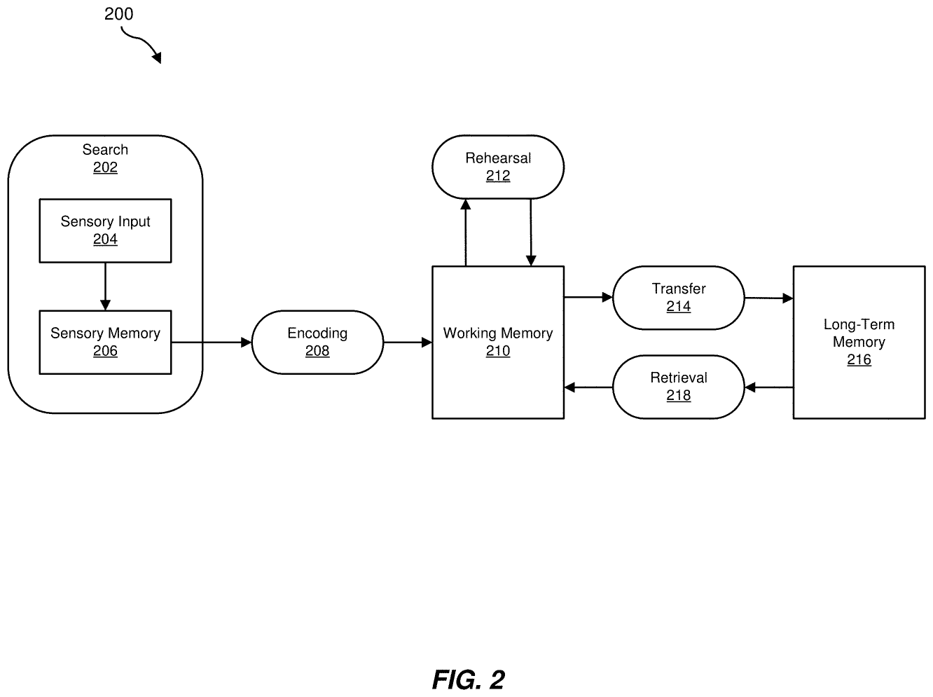

FIG. 1 is a block diagram of an example system 100 for signaling transitions between various cognitive states of users of example system 100. As illustrated in this figure, system 100 may include one or more modules 102 for performing one or more tasks. As will be explained in greater detail below, modules 102 may include an acquiring module 104 that acquires biosignals (e.g., eye-tracking signals indicative of gaze dynamics) generated by users of system 100. Example system 100 may also include a predicting module 106 that uses the biosignals acquired by acquiring module 104 to anticipate transitions (e.g., changes or switches) to, from, and/or between cognitive states of the users. For example, predicting module 106 may use biosignals acquired by acquiring module 104 to anticipate transitions to, from, and/or between any of the example cognitive states illustrated in FIG. 2. Example system 100 may further include a signaling module 108 that provides, to one or more intelligent-facilitation subsystems, signals indicating transitions between the cognitive states of the users.

As will be explained in greater detail below, the disclosed systems may anticipate transitions to, from, and/or between a variety of cognitive states. As used herein, the term “cognitive state” may refer to or include one or more cognitive tasks, functions, and/or processes involved in users acquiring knowledge and/or awareness through thinking, experiencing, and/or sensing. Additionally or alternatively, the term “cognitive state” may refer to or include one or more tasks, functions, and/or processes of cognition related to perceiving, concentrating, conceiving, remembering, reasoning, judging, comprehending, problem solving, and/or decision making. In some examples, the term “cognitive state” may refer to or include internal mental states that may not be externally observable.

FIG. 2 illustrates exemplary cognitive states 200 and their transitions. In this example, cognitive states 200 may include a search state 202 in which a user captures sensory input 204 from the user's senses into a sensory memory 206 of the user. In some examples, search state 202 may represent any cognitive state or task in which a user searches (e.g., visually searches) for a target stimuli (e.g., an entity in the user's environment, such as a thing, a person, or a condition, that may act as a stimulus and/or may elicit a response from the user) from among distractor stimuli presented to and/or previously memorized by the user. In some examples, sensory input 204 may represent or include any sensory information generated by a sensory organ of the user, and sensory memory 206 may represent or include a portion of the user's nervous system that briefly retains sensory information prior to being encoded into longer-term memory.

As shown in FIG. 2, cognitive states 200 may include an encoding state 208 in which sensory input 204 is converted into a form capable of being processed and deposited into a working memory 210 of the user. In some examples, working memory 210 may represent and/or include any short-term, temporary, or primary memory of the user. Cognitive states 200 may also include a rehearsal state 212 in which the user mentally repeats information stored to working memory 210 (e.g., in order to maintain it longer in working memory 210). Cognitive states may further include a transfer state 214 in which information from working memory 210 is transferred to and retained in a long-term memory 216 of the user. In some examples, long-term memory 216 may represent or include any long-term, permanent, or secondary memory of the user. Cognitive states 200 may additionally include a retrieval state 218 in which information stored in long-term memory 216 is located within long-term memory 216 and/or recovered to working memory 210.

Returning to FIG. 1, example system 100 may include one or more intelligent-facilitation subsystems (e.g., intelligent-facilitation subsystem(s) 101) that may respond or react to a user's cognitive-state transitions by performing one or more assistive actions or interventions that reduce the mental load, effort, or exertion associated with the involved cognitive states and/or any other associated cognitive states. In one example, intelligent-facilitation subsystem(s) 101 may respond to a transition to search state 202 by performing one or more assistive actions that reduce the user's mental load, effort, or exertion involved with search state 202. For example, intelligent-facilitation subsystem(s) 101 may reduce the user's mental load, effort, or exertion associated with search state 202 by presenting a list of often searched for items and/or their last recorded locations to the user in order to facilitate the user's search for the items. In another example, intelligent-facilitation subsystem(s) 101 may respond to a transition to encoding state 208, rehearsal state 212, and/or transfer state 214 by performing one or more assistive actions that reduce the mental load, effort, or exertion associated with encoding state 208, rehearsal state 212, and/or transfer state 214. For example, intelligent-facilitation subsystem(s) 101 may reduce the mental load, effort, or exertion associated with encoding state 208, rehearsal state 212, and/or transfer state 214 by presenting an assistive tool to the user that enables the user to record, to memory 120 for later retrieval, any information that the user is in the process of encoding, rehearsing, and/or transferring during encoding state 208, rehearsal state 212, and/or transfer state 214. As will be described in greater detail below, intelligent-facilitation subsystem(s) 101 may respond to cognitive-state transitions in a variety of additional ways.

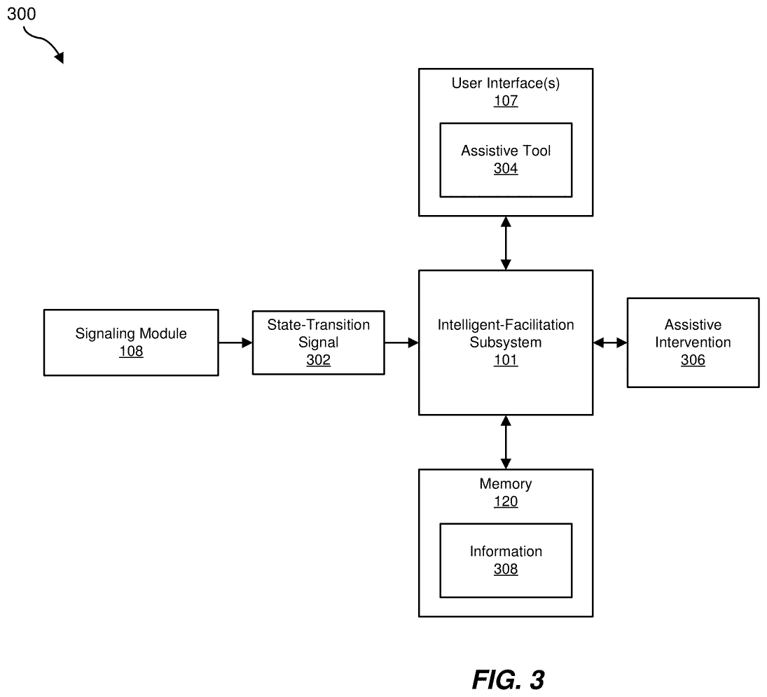

FIG. 3 illustrates an exemplary data flow 300 of intelligent-facilitation subsystem(s) 101 for intelligently facilitating a user's cognitive tasks and goals using adaptive interfaces and interventions in response to cognitive-state transitions. In this example, signaling module 108 may provide, to intelligent-facilitation subsystem 101, a state-transition signal 302 indicating the onset or occurrence of a cognitive-state transition. In some examples, intelligent-facilitation subsystem 101 may react to state-transition signal 302 by using user interface(s) 107 to present an assistive tool 304 to the user that intelligently facilitates a current or future cognitive state of the user (e.g., by facilitating the collection of information 308 from the user). In some examples, information 308 may represent, include, and/or be related to information that has been or is being encoded into the user's working memory and/or transferred to the user's long-term memory. Additionally or alternatively, intelligent-facilitation subsystem 101 may react to state-transition signal 302 by performing one or more assistive interventions 306 that store information 308 to memory 120 with or without the user's knowledge. For example, intelligent-facilitation subsystem 101 may react to state-transition signal 302 by gathering information about the user and/or the user's environment that the user may access at a later time when needed.

In some examples, assistive tool 304 may represent or include any tool that reduces the mental load, effort, or exertion associated with a current or future cognitive state of the user. Assistive tool 304 may include or represent a notepad, a list, a shopping list, a grocery list, a to-do list, a list of reminders, a journal, a diary, a catalog, an inventory, a calendar, a contact manager, a wallet, a sketchpad, a photo tool, a video tool, an audio tool, a map, an e-commerce tool, a user-input tool that facilitates the collection of information from the user, an information management tool that facilitates the search for and/or the retrieval of information stored to memory 120, variations or combinations of one or more of the same, or any other type or form of tool that may assist a user's cognitive tasks and/or goals. In some examples, assistive intervention 306 may include or represent any action or process that facilitates assistive tool 304.

Returning again to FIG. 1, example system 100 may include one or more sensors (e.g., biosensor(s) 103 and/or environmental sensor(s) 105) for acquiring information about users of example system 100 and/or their environments. In some embodiments, biosensor(s) 103 may represent or include one or more physiological sensors capable of generating real-time biosignals indicative of one or more physiological characteristics of users and/or for making real-time measurements of biopotential signals generated by users. A physiological sensor may represent or include any sensor that detects or measures a physiological characteristic or aspect of a user (e.g., gaze, heart rate, respiration, perspiration, skin temperature, body position, and so on). In some embodiments, biosensor(s) 103 may collect, receive, and/or identify biosensor data that indicates, either directly or indirectly, physiological information that may be associated with and/or help identify users' cognitive-state transitions. In some examples, biosensor(s) 103 may represent or include one or more human-facing sensors capable of measuring physiological characteristics of users. Examples of biosensor(s) 103 include, without limitation, eye-tracking sensors, hand-tracking sensors, body-tracking sensors, heart-rate sensors, cardiac sensors, neuromuscular sensors, electrooculography (EOG) sensors, electromyography (EMG) sensors, electroencephalography (EEG) sensors, electrocardiography (ECG) sensors, microphones, visible light cameras, infrared cameras, ambient light sensors (ALSs), inertial measurement units (IMUs), heat flux sensors, temperature sensors configured to measure skin temperature, humidity sensors, bio-chemical sensors, touch sensors, proximity sensors, biometric sensors, saturated-oxygen sensors, biopotential sensors, bioimpedance sensors, pedometer sensors, optical sensors, sweat sensors, variations or combinations of one or more of the same, or any other type or form of biosignal-sensing device or system.

In some embodiments, environmental sensor(s) 105 may represent or include one or more sensing devices capable of generating real-time signals indicative of one or more characteristics of users' environments. In some embodiments, environmental sensor(s) 105 may collect, receive, and/or identify data that indicates, either directly or indirectly, an entity in the user's environment, such as a thing, a person, or a condition, that a user may wish to interact with and/or remember. Examples of environmental sensor(s) 105 include, without limitation, cameras, microphones, Simultaneous Localization and Mapping (SLAM) sensors, Radio-Frequency Identification (RFID) sensors, variations or combinations of one or more of the same, or any other type or form of environment-sensing or object-sensing device or system.

As further illustrated in FIG. 1, example system 100 may also include one or more transition-predicting models, such as transition-predicting model(s) 140, trained and/or otherwise configured to predict cognitive-state transitions and/or otherwise model cognitive states using biosignal information. In at least one embodiment, transition-predicting model(s) 140 may include or represent a gazed-based predictive model that takes as input information indicative of gaze dynamics and/or eye movements and outputs a prediction (e.g., a probability or binary indicator) of one or more cognitive-state transitions. In some embodiments, the disclosed systems may train transition-predicting model 140 to make real-time predictions of users' cognitive-state transitions, decode moments of transitioning between cognitive states from gaze data, and/or predict the temporal onset of cognitive states. In some embodiments, the disclosed systems may train transition-predicting model 140 to predict the temporal onset of a transition between cognitive states using nothing more than gaze dynamics leading up to the moment of the transition. In at least one example, the disclosed systems may train transition-predicting model 140 to predict the temporal onset of cognitive-state transitions using only eye-tracking data that preceded transition events.

Transition-predicting model(s) 140 may represent or include any machine-learning model, algorithm, heuristic, data, or combination thereof, that may anticipate, recognize, detect, estimate, predict, label, infer, and/or react to the temporal onset of a user's cognitive-state transitions based on and/or using biosignals acquired from one or more biosensors, such as biosensors 103. Examples of transition-predicting model(s) 140 include, without limitation, decision trees (e.g., boosting decision trees), neural networks (e.g., a deep convolutional neural network), deep-learning models, support vector machines, linear classifiers, non-linear classifiers, perceptrons, naive Bayes classifiers, any other machine-learning or classification techniques or algorithms, or any combination thereof.

The systems describe herein may train transition-predicting models, such as transition-predicting model 140, to predict the timing of cognitive-state transitions in any suitable way. In one example, the systems may train a transition-predicting model to predict when a user is starting to and/or about to transition between two cognitive states using a ground-truth time series of physiological data that includes physiological data recorded before and/or up to the transition between the two cognitive states. In some examples, the time series may include samples preceding a user's transition between two cognitive states by approximately 10 ms, 50 ms, 100 ms, 200 ms, 300 ms, 400 ms, 500 ms, 600 ms, 700 ms, 800 ms, 900 ms, 1000 ms, 1100 ms, 1200 ms, 1300 ms, 1400 ms, 1500 ms, 1600 ms, 1700 ms, 1800 ms, 1900 ms, or 2000 ms. Additionally or alternatively, the time series include samples preceding a transition between two cognitive states by approximately 2100 ms, 2200 ms, 2300 ms, 2400 ms, 2500 ms, 2600 ms, 2700 ms, 2800 ms, 2900 ms, 3000 ms, 3100 ms, 3200 ms, 3300 ms, 3400 ms, 3500 ms, 3600 ms, 3700 ms, 3800 ms, 3900 ms, 4000 ms, 4100 ms, 4200 ms, 4300 ms, 4400 ms, 4500 ms, 4600 ms, 4700 ms, 4800 ms, 4900 ms, 5000 ms, 5100 ms, 5200 ms, 5300 ms, 5400 ms, 5500 ms, 5600 ms, 5700 ms, 5800 ms, 5900 ms, 6000 ms, 6100 ms, 6200 ms, 6300 ms, 6400 ms, 6500 ms, 6600 ms, 6700 ms, 6800 ms, 6900 ms, 7000 ms, 7100 ms, 7200 ms, 7300 ms, 7400 ms, 7500 ms, 7600 ms, 7700 ms, 7800 ms, 7900 ms, 8000 ms, 8100 ms, 8200 ms, 8300 ms, 8400 ms, 8500 ms, 8600 ms, 8700 ms, 8800 ms, 8900 ms, 9000 ms, 9100 ms, 9200 ms, 9300 ms, 9400 ms, 9500 ms, 9600 ms, 9700 ms, 9800 ms, 9900 ms, 10000 ms, 10100 ms, 10200 ms, 10300 ms, 10400 ms, 10500 ms, 10600 ms, 10700 ms, 10800 ms, or 10900 ms. In some embodiments, a transition-predicting model may take as input a similar time series of physiological data.

In some embodiments, the disclosed systems may use one or more transition-predicting models (e.g., a transition-predicting model trained for an individual user or a transition-predicting model trained for a group of users). In at least one embodiment, the disclosed systems may train models to make predictions for cognitive-state transitions that are on the scale of milliseconds or seconds.

As further illustrated in FIG. 1, example system 100 may also include one or more memory devices, such as memory 120. Memory 120 may include or represent any type or form of volatile or non-volatile storage device or medium capable of storing data and/or computer-readable instructions. In one example, memory 120 may store, load, and/or maintain one or more of modules 102. Examples of memory 120 include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

As further illustrated in FIG. 1, example system 100 may also include one or more physical processors, such as physical processor 130. Physical processor 130 may include or represent any type or form of hardware-implemented processing unit capable of interpreting and/or executing computer-readable instructions. In one example, physical processor 130 may access and/or modify one or more of modules 102 stored in memory 120. Additionally or alternatively, physical processor 130 may execute one or more of modules 102 to facilitate prediction or signaling of cognitive-state transitions. Examples of physical processor 130 include, without limitation, microprocessors, microcontrollers, central processing units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

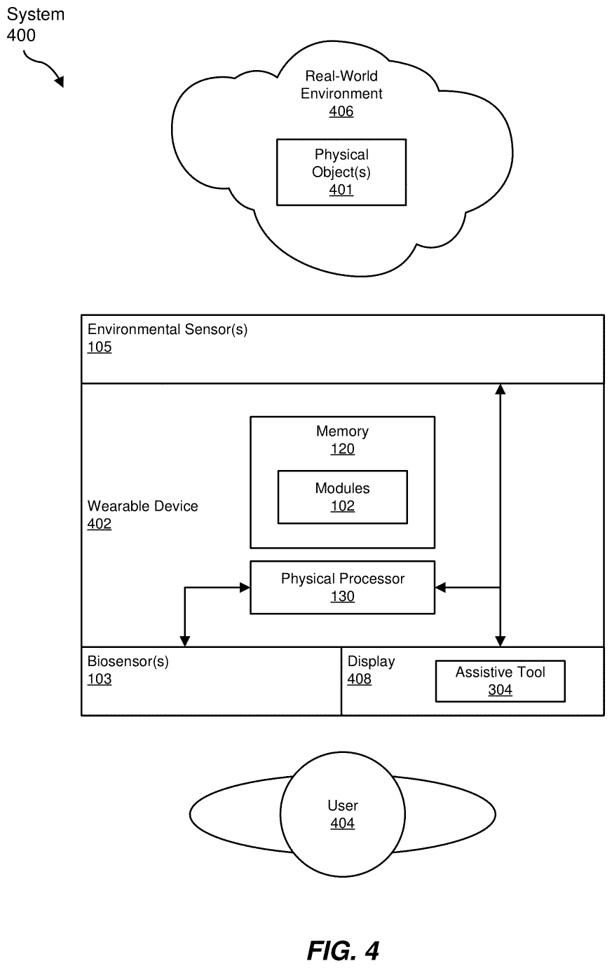

System 100 in FIG. 1 may be implemented in a variety of ways. For example, all or a portion of system 100 may represent portions of an example system 400 in FIG. 4. As shown in FIG. 4, system 400 may include a wearable device 402 (e.g., a wearable XR device) having (1) one or more user-facing sensors (e.g., biosensor(s) 103) capable of acquiring biosignal data generated by a user 404, (2) one or more environment-facing sensors (e.g., environmental sensor(s) 105) capable of acquiring environmental data about a real-world environment 406 of user 404, and/or (3) a display 408 capable of displaying assistive tools to user 404.

As shown in FIG. 4, wearable device 402 may be programmed with one or more of modules 102 from FIG. 1 (e.g., acquiring module 104, predicting module 106, and/or signaling module 108) that may, when executed by wearable device 402, enable wearable device 402 to (1) acquire, via one or more of biosensor(s) 103, one or more biosignals generated by user 404, (2) use the one or more biosignals to anticipate transitions to, from, and/or between cognitive states of user 404, and (3) provide a state-transition signal indicating the transitions to, from, and/or between cognitive states of user 404 to an intelligent-facilitation subsystem of wearable device 402.

FIG. 5 is a flow diagram of an exemplary computer-implemented method 500 for signaling cognitive-state transitions. The steps shown in FIG. 5 may be performed by any suitable computer-executable code and/or computing system, including the system(s) illustrated in FIGS. 1-4 and 11-20. In one example, each of the steps shown in FIG. 5 may represent an algorithm whose structure includes and/or is represented by multiple sub-steps, examples of which will be provided in greater detail below.

As illustrated in FIG. 5, at step 510 one or more of the systems described herein may acquire, via one or more biosensors, one or more biosignals generated by a user of a computing system. For example, acquiring module 104 may, as part of wearable device 402 in FIG. 4, use one or more of biosensors 103 to acquire one or more raw and/or derived biosignals generated by user 404.

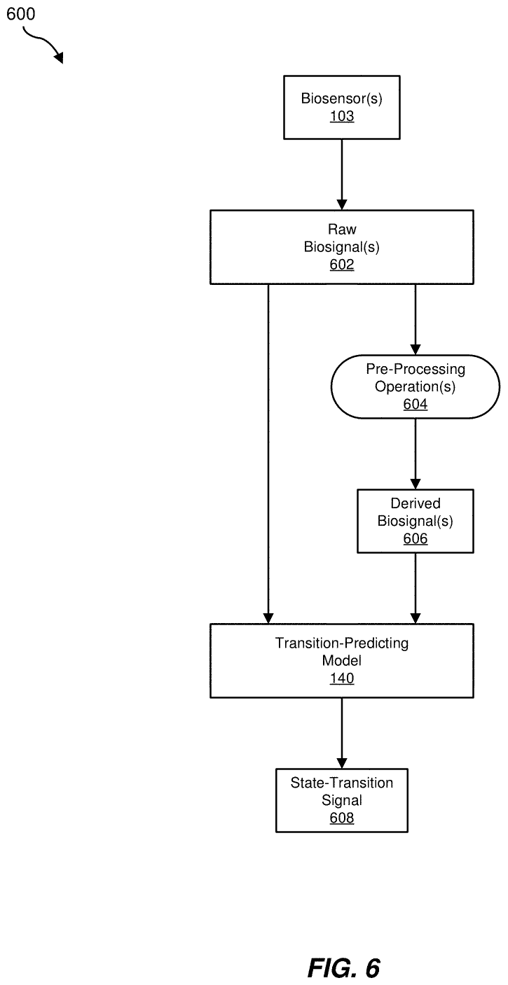

The systems described herein may perform step 510 in a variety of ways. FIG. 6 illustrates an exemplary data flow 600 for acquiring biosignal data and using the biosignal data to generate transition signals. As shown in this figure, in some embodiments, the disclosed systems may receive raw biosignal(s) 602 from biosensor(s) 103 and may use raw biosignal(s) 602 as input to transition-predicting model 140. Additionally or alternatively, the disclosed systems may generate one or more derived biosignal(s) 606 by performing one or more pre-processing operation(s) 604 (e.g., event-detection or feature-extraction operations) on raw biosignal(s) 602 and then may use derived biosignal(s) 606 as input to transition-predicting model 140.

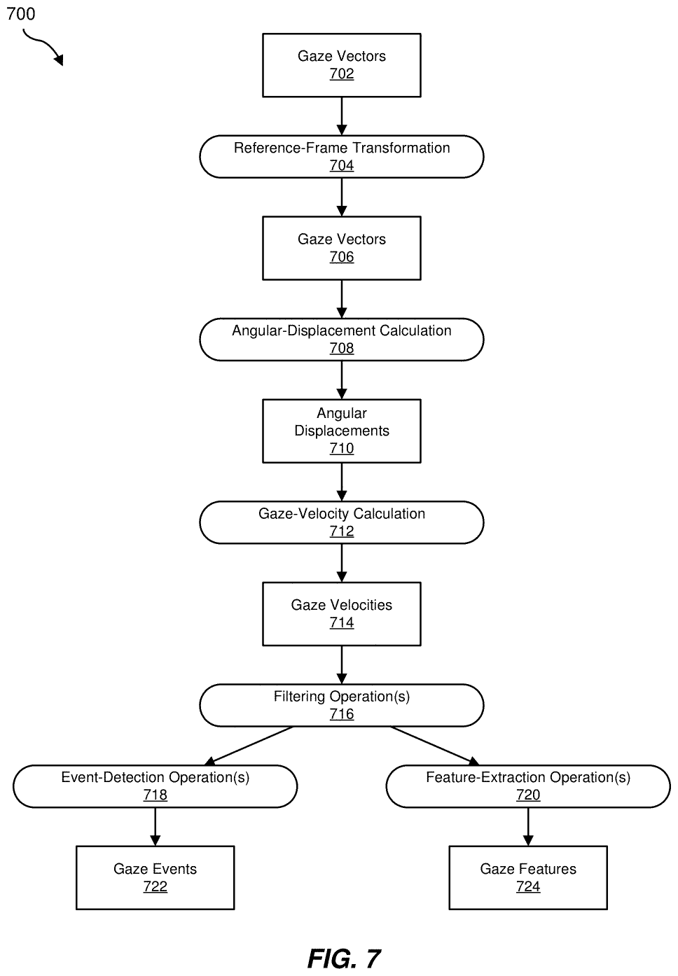

FIG. 7 illustrates an exemplary real-time pre-processing pipeline 700 that may be used by the disclosed systems to transform raw, real-time eye-tracking data into one or more of the features disclosed herein from which a user's cognitive-state transitions may be anticipated. In this example, the disclosed systems may acquire a stream of real-time, 3D gaze vectors 702 from an eye-tracking system. In some examples, 3D gaze vectors 702 may be in an eye-in-head frame of reference, and the disclosed systems may transform 3D gaze vectors 702 to an eye-in-world frame of reference using a suitable reference-frame transformation 704 (e.g., using information indicating the user's head orientation), which may result in transformed 3D gaze vectors 706. Next, the disclosed systems may compute angular displacements 710 between consecutive samples from gaze vectors 706 using a suitable angular-displacement calculation 708. For example, the disclosed system may compute angular displacements 710 between consecutive samples from gaze vectors 706 using Equation (1):

θ=2×a tan 2(∥u−v∥,∥u+v∥) (1)

where consecutive samples of gaze vectors 706 are represented as normalized vectors u and v and the corresponding angular displacement is represented as θ.

The disclosed systems may then calculate gaze velocities 714 from angular displacements 710 using a suitable gaze-velocity calculation 712. For example, the disclosed systems may divide each sample from angular displacements 710 (e.g., θ, as calculated above) by the change in time between associated consecutive samples from gaze vectors 706.

In some embodiments, the disclosed systems may perform one or more filtering operation(s) 716 on gaze velocities 712 (e.g., to remove noise and/or unwanted segments before downstream event detection and feature extraction). In at least one embodiment, the disclosed systems may remove all samples where gaze velocity exceeds about 800 degrees/second, which may indicate unfeasibly fast eye movements for general users. In another embodiment, the disclosed systems may remove all samples where gaze velocity exceeds about 1000 degrees/second, which may alternatively indicate unfeasibly fast eye movements for certain groups of users (e.g., younger users). The disclosed systems may then replace removed values through interpolation. Additionally or alternatively, the disclosed systems may apply a median filter (e.g., a median filter with a width of seven samples) to gaze velocities 714 to smooth the signal and/or account for noise.

In some embodiments, the disclosed systems may generate gaze events 722 from gaze velocities 714 by performing one or more event-detection operation(s) 718. In some embodiments, the disclosed systems may detect fixation events (e.g., moments of maintaining visual gaze on a single location) and/or saccade events (e.g., moments of rapid eye movement between points of fixation) from gaze velocities 714 using any suitable detection model, algorithm, or heuristic. For example, the disclosed systems may perform saccade detection using a suitable saccade detection algorithm (e.g., Velocity-Threshold Identification (I-VT), Dispersion-Threshold Identification (I-DT), or Hidden Markov Model Identification (I-HMM)). In at least one embodiment, the disclosed systems may perform I-VT saccade detection by identifying consecutive samples from gaze velocities 714 that exceeded about 70 degrees/second. In some embodiments, the disclosed systems may require a minimum duration in the range of about 5 milliseconds to about 30 milliseconds (e.g., 17 milliseconds) and a maximum duration in the range of about 100 milliseconds to about 300 milliseconds (e.g., 200 milliseconds) for saccade events. In some embodiments, the disclosed systems may perform I-DT fixation detection by computing dispersion (e.g., the largest angular displacement from the centroid of gaze samples) over predetermined time windows and marking time windows where dispersion did not exceed about 1 degree as fixation events. In some embodiments, the disclosed systems may require a minimum duration in the range of about 50 milliseconds to about 200 milliseconds (e.g., 100 milliseconds) and a maximum duration in the range of about 0.5 seconds to about 3 seconds (e.g., 2 seconds) for fixation events.

In some embodiments, the disclosed systems may generate gaze features 724 by performing one or more event-extraction operation(s) 720 on gaze vectors 702, gaze vectors 706, angular displacements 710, gaze velocities 714, and/or any other suitable eye-tracking data. The disclosed systems may extract a variety of gaze-based features for use in predicting cognitive-state transitions with a computing system. Examples of gaze-based features include, without limitation, gaze velocity (e.g., a measure of how fast gaze is moving), ambient attention, focal attention, saccade dynamics, gaze features that characterize visual attention, dispersion (e.g., a measure of how spread out gaze points are over a period of time), event-detection labels, low-level eye movement features derived from gaze events 722, the K coefficient (e.g., to discern between focal and ambient behavior), variations or combinations of one or more of the same, or any other type or form of eye-tracking data.

The systems described herein may predict when a cognitive-state transition occurs using a variety of gaze data and gaze dynamics. For example, the disclosed systems may predict moments of cognitive-state transitions using a combination of gaze velocity, low-level features from fixation and saccade events, and/or mid-level features that recognize patterns in the shape of scan paths. In some embodiments, the systems described herein may predict a user's cognitive-state transitions based on patterns and/or elements of one or more of fixation events (e.g., whether or not a user is fixated on something), gaze velocity, fixation average velocity, saccade acceleration skew in the x direction, saccade standard deviation in the y direction, saccade velocity kurtosis, saccade velocity skew, saccade velocity skew in the y direction, saccade duration, ambient/focal K coefficient, saccade velocity standard deviation, saccade distance from previous saccade, dispersion, fixation duration, fixation kurtosis in the y direction, saccade velocity kurtosis in the x direction, saccade velocity skew in the x direction, saccade amplitude, saccade standard deviation in the x direction, fixation kurtosis in the x direction, saccade acceleration kurtosis in the y direction, saccade acceleration skew, fixation skew in the y direction, saccade acceleration kurtosis in the x direction, saccade events (e.g., whether or not a user is performing a saccade), saccade dispersion, fixation standard deviation in the x direction, fixation skew in the x direction, saccade velocity mean, fixation standard deviation in the y direction, saccade velocity kurtosis in the y direction, fixation angle from previous fixation, saccade angle from previous saccade, saccade velocity median in the x direction, fixation path length, saccade acceleration skew in the y direction, fixation dispersion, saccade acceleration kurtosis, saccade path length, saccade acceleration median in the y direction, saccade velocity mean in the x direction, saccade acceleration median in the y direction, saccade velocity mean in the x direction, saccade acceleration standard deviation in the x direction, saccade velocity mean in the y direction, saccade acceleration mean, saccade acceleration mean in the x direction, saccade acceleration median in the x direction, saccade acceleration standard deviation, saccade acceleration standard deviation in the y direction, saccade velocity standard deviation in the y direction, saccade acceleration maximum in the x direction, saccade velocity median, saccade velocity maximum in the x direction, saccade acceleration maximum, saccade acceleration median, saccade velocity median in the y direction, saccade acceleration mean in the y direction, saccade ratio, saccade velocity standard deviation in the x direction. Additionally or alternatively, the systems described herein may predict a user's cognitive-state transitions based on gaze velocity, any suitable measure of ambient/focal attention, statistical features of saccadic eye movements, blink patterns, scan path patterns, and/or changes to pupil features.

Returning to FIG. 5 at step 520, one or more of the systems described herein may use the one or more biosignals acquired at step 510 to anticipate a transition to or from a cognitive state of a user. For example, predicting module 106 may use, as part of wearable device 402, one or more of biosignals 602 and/or 606 to anticipate a transition to or from a cognitive state of user 404. The systems described herein may perform step 520 in a variety of ways. In one example, the disclosed systems may use a suitably trained predictive model (e.g., transition-predicting model 140) to predict the onset of cognitive-state transitions.

At step 530 one or more of the systems described herein may provide a signal indicating the cognitive-state transitions anticipated at step 520 to an intelligent-facilitation subsystem. For example, signaling module 108 may, as part of wearable device 402 in FIG. 4, provide a signal indicating a cognitive-state transition of user 404 to intelligent-facilitation subsystem(s) 101.

The systems described herein may perform step 530 in a variety of ways. In some examples, the disclosed systems may use publish/subscribe messaging to exchange signals of cognitive state transitions. For example, signaling module 108 may publish (e.g., using a suitable application programming interface, multiple signal types each signaling a certain type of cognitive-state transition to which intelligent-facilitation subsystem(s) 101 (e.g., third-party applications) may subscribe and react. In at least one example, the disclosed systems may include a variety of information about a cognitive-state transition in a state-transition signal. For example, the disclosed systems may indicate a type of the cognitive-state transition, the cognitive states involved in the transition, the timing of the cognitive-state transition, a probability or likelihood of the cognitive-state transition, a context (e.g., environmental context) in which the cognitive-state transition is occurring, and/or any other information that may be helpful to an intelligent-facilitation subsystem in reacting to a user's cognitive-state transitions.

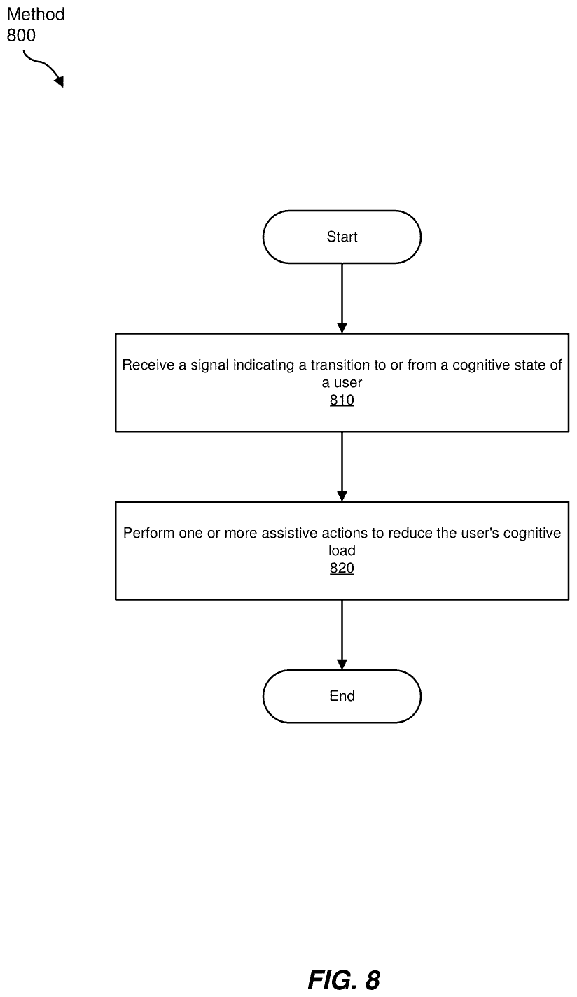

The disclosed intelligent-facilitation subsystems may respond and/or react to state-transition signals in a variety of ways. FIG. 8 is a flow diagram of an exemplary computer-implemented method 800 for responding to and/or reacting to cognitive-state transitions. The steps shown in FIG. 8 may be performed by any suitable computer-executable code and/or computing system, including the system(s) illustrated in FIGS. 1-4 and 11-20. In one example, each of the steps shown in FIG. 8 may represent an algorithm whose structure includes and/or is represented by multiple sub-steps.

As illustrated in FIG. 8, at step 810 one or more of the systems described herein may receive a signal indicating a transition to or from a cognitive state of a user. For example, intelligent-facilitation subsystem 101 may, as part of wearable device 402 in FIG. 4, receive a signal indicating a transition to or from a cognitive state of user 404. At step 820, one or more of the systems described herein may perform, in response to the signal received at step 810, one or more assistive actions to reduce the user's cognitive load. For example, intelligent-facilitation subsystem 101 may, as part of wearable device 402 in FIG. 4, display assistive tool 304 to user 404 and/or perform one or alternative or additional assistive actions to reduce the current and/or future cognitive loads of user 404.

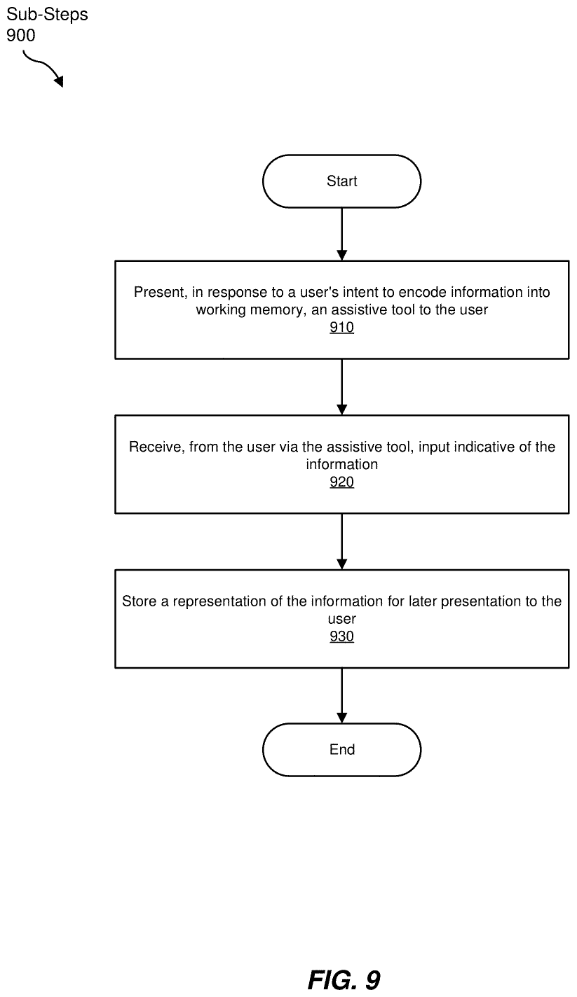

The systems described herein may perform step 820 in a variety of ways. FIG. 9 is a flow diagram of exemplary sub-steps 900 for performing assistive actions in response to signals indicating a user's intent to encode information to working memory. At sub-step 910, one or more of the systems described herein may identify and present an appropriate assistive tool, such as those described above, to the user to facilitate the user in providing the encoded information to the system for duplicative storage in machine memory (e.g., memory 120). In some examples, the disclosed systems may automatically identify and present an assistive tool appropriate to the user's current cognitive state, task, or goal without requiring the user to explicitly request the assistive tool. At sub-step 920, one or more of the systems described herein may use the assistive tool to receive input from the user that indicates the encoded information. In at least one embodiment, the disclosed systems may assist the user in providing the information by presenting a list of possibilities to the user. Then at sub-step 930, one or more of the disclosed systems may store a representation of the information to machine memory for later retrieval by and/or presentation to the user (e.g., in response to a signal indicating a transition to a retrieval state).

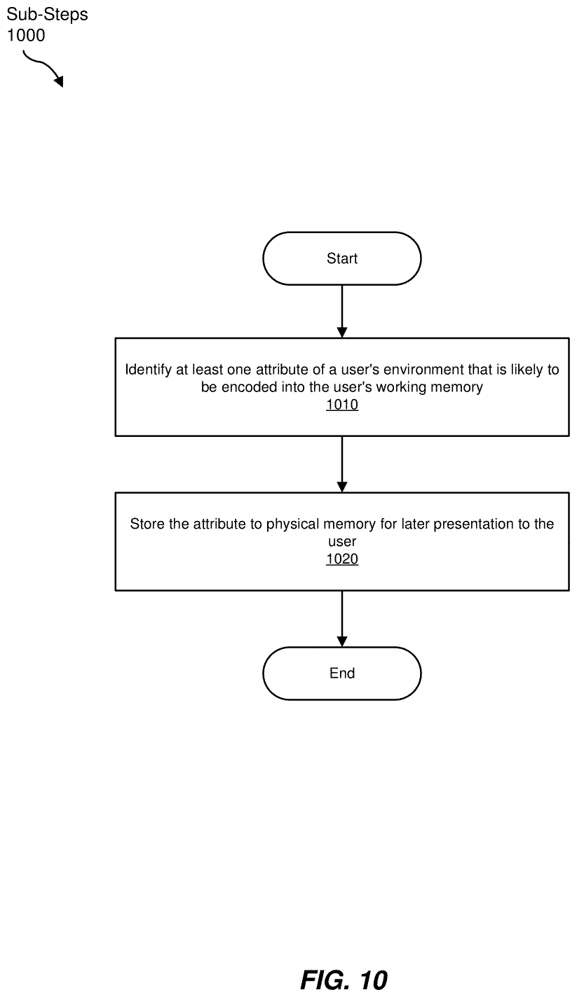

FIG. 10 is a flow diagram of additional exemplary sub-steps 1000 for performing assistive actions in response to signals indicating a user's intent to encode information to working memory. At sub-step 1010, one or more of the systems described herein may identify at least one attribute of the user's environment that is likely to be encoded into the user's working memory. For example, the disclosed systems may identify attributes of a location of the user's environment, attributes of entities (e.g., objects, people, dates, addresses, vocabulary, or images) in the environment, attributes of new entities in the environment that have not been encountered before, or attributes of missing entities that were previously in the environment. In some examples, the disclosed systems may identify attributes of the user's environment with help from the user (e.g., via an assistive tool) and/or without the user's help or knowledge. At sub-step 1020, one or more of the systems described herein may store the attribute to physical memory for later presentation to the user (e.g., in response to a signal indicating a transition to a retrieval state).

In some examples, the disclosed systems may gather and/or record information about the context of a cognitive-state transition or a previous cognitive-state transition in order to determine what assistive tools and/or interventions might best help a user. In some examples, the disclosed systems may determine an appropriate assistive tool or intervention based on (1) information about the environment of the cognitive-state transition (e.g., the location of the environment and/or items previously and/or currently within the environment), (2) information about the user's prior and/or current movements within the environment, (3) information about the timing of the cognitive-state transition, (4) information about the user's focal attention before, during, or after the cognitive transition, and/or (5) information about the user's prior uses of assistive tools.

In one non-limiting example, the disclosed systems may present a grocery list to a user after determining that the user is likely encoding grocery items into working memory (e.g., based on a transition to an encoding state of the user occurring in the user's kitchen). In another non-limiting example, the disclosed systems may present a contact list or a communication tool to a user after determining that the user is likely encoding contact information into working memory (e.g., based on the detection of contact information, such as a phone number, in the user's field of view). In another non-limiting example, the disclosed systems may present a digital wallet or another form of payment information to a user after determining that the user is likely trying to encode payment information (e.g., based on the focal attention of the user being directed at a credit card number during an encoding state) and/or after determining that the user is likely trying to recall payment information (e.g., based on detecting that the user is on a payment page of an e-commerce website during a retrieval state). In some examples, the disclosed systems may reduce the friction of filling in payment information by filling in the payment information automatically and/or by enabling the user to fill in the payment information with a single action such as a single click.

In another non-limiting example, the disclosed systems may present a dictionary to a user after determining that the user is likely trying to retrieve a definition of a word (e.g., based on the focal attention of the user being directed at the word during a retrieval state). In other non-limiting examples, the disclosed systems may present an address book to a user after determining that the user is likely trying to encode an address (e.g., based on the focal attention of the user being directed at the address during an encoding state) and/or after determining that the user is likely trying to recall an address from the user's long-term memory (e.g., based on the focal attention of the user being directed at an address form during a retrieval state). In another non-limiting example, the disclosed systems may present an item (e.g., an instruction manual) previously accessed by a user after determining that the user is in an environment or situation that is similar to the environment or situation in which the user last accessed the item and the user is trying to retrieve information from long-term memory.

In some non-limiting examples, the disclosed systems may automatically store contact information (e.g., names, titles, photos, or event details) for a user after determining that the user is engaging a previously unknown person during an encoding state). Later when the user is in the same persons presence and in a retrieval state, the disclosed systems may automatically present the stored contact information to the user. In another non-limiting example, the disclosed systems may automatically store a new vocabulary word to a dictionary after determining that the user is likely trying to encode the new vocabulary word (e.g., based on the focal attention of the user being directed at the word during a rehearsal state).

In another non-limiting example, the disclosed systems may automatically create a meeting, an appointment, or a reminder in a calendar tool after determining that the user is likely encoding information about an event (e.g., by detecting details of the event, such as a date or time, within the user's field of view during an encoding state). In another non-limiting example, the disclosed systems may automatically track details about the items that a user often searches for (e.g., keys, glasses, and phones) by noting items in the user's field of view or possession during a transition from a search state. The disclosed system may later automatically provide details about the items (e.g., when the user is in a retrieval state at a similar location or time and the items are not in the user's possession).

### Example Embodiments

Example 1: A computer-implemented method may include (1) acquiring, via one or more biosensors, one or more biosignals generated by a user of a computing system, (2) using the one or more biosignals to anticipate a transition to or from a cognitive state of the user, and (3) providing a signal indicating the transition to or from the cognitive state of the user to an intelligent-facilitation subsystem adapted to perform one or more assistive actions to reduce the user's cognitive load. In some embodiments, the computing system may include the intelligent-facilitation subsystem.

Example 2: The computer-implemented method of Example 1, wherein the steps of acquiring, using, and providing are performed when the user is not attentively engaged with the computing system.

Example 3: The computer-implemented method of any of Examples 1-2, wherein (1) the one or more biosensors include one or more eye-tracking sensors, (2) the one or more biosignals include signals indicative of gaze dynamics of the user, and (3) the signals indicative of gaze dynamics of the user are used to anticipate the transition to or from the cognitive state of the user.

Example 4: The computer-implemented method of any of Examples 1-3, wherein the signals indicative of gaze dynamics of the user include a measure of gaze velocity.

Example 5: The computer-implemented method of any of Examples 1-4, wherein the signals indicative of gaze dynamics of the user include a measure of ambient attention and/or a measure of focal attention.

Example 6: The computer-implemented method of any of Examples 1-5, wherein the signals indicative of gaze dynamics of the user include a measure of saccade dynamics.

Example 7: The computer-implemented method of any of Examples 1-6, wherein the cognitive state of the user includes a state of encoding information to working memory of the user.

Example 8: The computer-implemented method of any of Examples 1-7, wherein the cognitive state of the user includes a state of visual searching.

Example 9: The computer-implemented method of any of Examples 1-8, wherein the cognitive state of the user includes a state of storing information to long-term memory of the user.

Example 10: The computer-implemented method of any of Examples 1-9, wherein the cognitive state of the user includes a state of retrieving information from long-term memory of the user.

Example 11: The computer-implemented method of any of Examples 1-10, further including (1) receiving, by the intelligent-facilitation subsystem, the signal indicating the transition to or from the cognitive state of the user and (2) performing, by the intelligent-facilitation subsystem, the one or more assistive actions to reduce the user's cognitive load.

Example 12: The computer-implemented method of any of Examples 1-11, wherein (1) using the one or more biosignals to anticipate the transition to or from the cognitive state of the user includes using the one or more biosignals to anticipate the user's intent to encode information into working memory of the user and (2) performing the one or more assistive actions to reduce the user's cognitive load includes (a) presenting, to the user, at least one of a virtual notepad, a virtual list, and/or a virtual sketchpad, (b) receiving, from the user, input indicative of the information, and (3) storing, by the intelligent-facilitation subsystem, a representation of the information for later retrieval and presentation to the user.

Example 13: The computer-implemented method of any of Examples 1-12, wherein (1) the computing system includes physical memory and (2) performing the one or more assistive actions to reduce the user's cognitive load includes (a) identifying, by the intelligent-facilitation subsystem, at least one attribute of the user's environment that is likely to be encoded into the user's working memory and (b) storing the attribute to the physical memory for later presentation to the user.

Example 14: The computer-implemented method of any of Examples 1-13, wherein the intelligent-facilitation subsystem refrains from identifying the at least one attribute of the user's environment until after receiving the signal indicating the transition to or from the cognitive state of the user.

Example 15: A system may include (1) an intelligent-facilitation subsystem adapted to perform one or more assistive actions to reduce a user's cognitive load, (2) one or more biosensors adapted to detect biosignals generated by the user, (3) at least one physical processor, and (4) physical memory including computer-executable instructions that, when executed by the physical processor, cause the physical processor to (a) acquire, via the one or more biosensors, one or more biosignals generated by the user, (b) use the one or more biosignals to anticipate a transition to or from a cognitive state of the user, and (c) provide, to the intelligent-facilitation subsystem, a signal indicating the transition to or from the cognitive state of the user.

Example 16: The system of Example 15, wherein (1) the one or more biosensors include one or more eye-tracking sensors adapted to measure gaze dynamics of the user, (2) the one or more biosignals include signals indicative of the gaze dynamics of the user, and (3) the gaze dynamics of the user are used to anticipate the transition to or from the cognitive state of the user.

Example 17: The system of any of Examples 15-16, wherein (1) the one or more biosensors include one or more hand-tracking sensors, (2) the one or more biosignals include signals indicative of hand dynamics of the user, and (3) the signals indicative of hand dynamics of the user are used to anticipate the transition to or from the cognitive state of the user.

Example 18: The system of any of Examples 15-17, wherein (1) the one or more biosensors include one or more neuromuscular sensors, (2) the one or more biosignals include neuromuscular signals obtained from the user's body, and (3) the neuromuscular signals obtained from the user's body are used to anticipate the transition to or from the cognitive state of the user.

Example 19: The system of any of Examples 15-18, wherein (1) the system is an extended-reality system and (2) the intelligent-facilitation subsystem is further adapted to (a) receive the signal indicating the transition to or from the cognitive state of the user and (b) perform, in response to receiving the signal, the one or more assistive actions to reduce the user's cognitive load.

Example 20: A non-transitory computer-readable medium may include one or more computer-executable instructions that, when executed by at least one processor of a computing device, cause the computing device to (1) acquire, via one or more biosensors, one or more biosignals generated by a user of the computing device, (2) use the one or more biosignals to anticipate a transition to or from a cognitive state of the user, and (3) provide a signal indicating the transition to or from the cognitive state of the user to an intelligent-facilitation subsystem adapted to perform one or more assistive actions to reduce the user's cognitive load.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

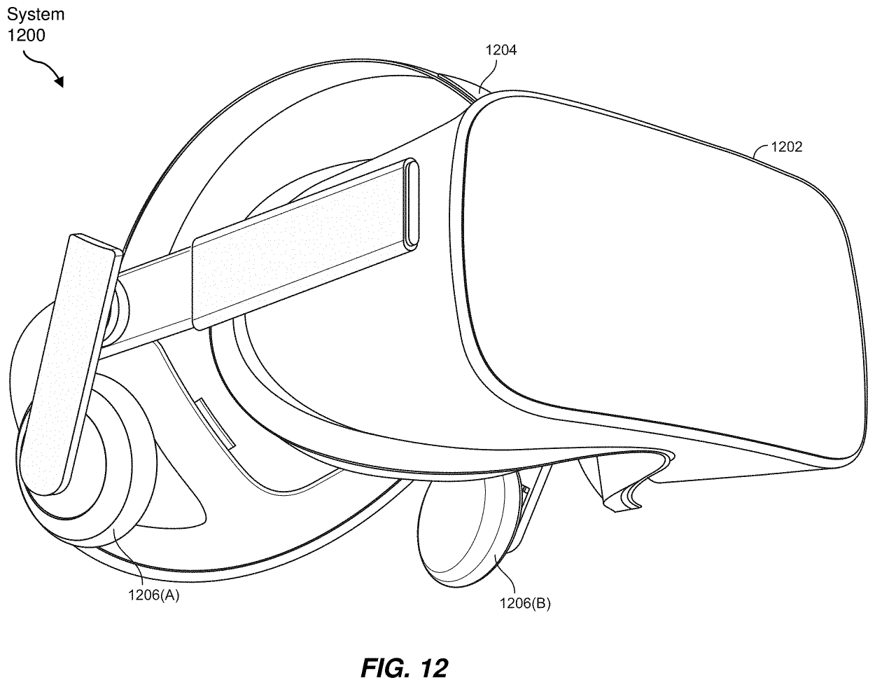

Artificial-reality systems may be implemented in a variety of different form factors and configurations. Some artificial-reality systems may be designed to work without near-eye displays (NEDs). Other artificial-reality systems may include an NED that also provides visibility into the real world (such as, e.g., augmented-reality system 1100 in FIG. 11) or that visually immerses a user in an artificial reality (such as, e.g., virtual-reality system 1200 in FIG. 12). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 11, augmented-reality system 1100 may include an eyewear device 1102 with a frame 1110 configured to hold a left display device 1115(A) and a right display device 1115(B) in front of a user's eyes. Display devices 1115(A) and 1115(B) may act together or independently to present an image or series of images to a user. While augmented-reality system 1100 includes two displays, embodiments of this disclosure may be implemented in augmented-reality systems with a single NED or more than two NEDs.

In some embodiments, augmented-reality system 1100 may include one or more sensors, such as sensor 1140. Sensor 1140 may generate measurement signals in response to motion of augmented-reality system 1100 and may be located on substantially any portion of frame 1110. Sensor 1140 may represent one or more of a variety of different sensing mechanisms, such as a position sensor, an inertial measurement unit (IMU), a depth camera assembly, a structured light emitter and/or detector, or any combination thereof. In some embodiments, augmented-reality system 1100 may or may not include sensor 1140 or may include more than one sensor. In embodiments in which sensor 1140 includes an IMU, the IMU may generate calibration data based on measurement signals from sensor 1140. Examples of sensor 1140 may include, without limitation, accelerometers, gyroscopes, magnetometers, other suitable types of sensors that detect motion, sensors used for error correction of the IMU, or some combination thereof.

In some examples, augmented-reality system 1100 may also include a microphone array with a plurality of acoustic transducers 1120(A)-1120(J), referred to collectively as acoustic transducers 1120. Acoustic transducers 1120 may represent transducers that detect air pressure variations induced by sound waves. Each acoustic transducer 1120 may be configured to detect sound and convert the detected sound into an electronic format (e.g., an analog or digital format). The microphone array in FIG. 11 may include, for example, ten acoustic transducers: 1120(A) and 1120(B), which may be designed to be placed inside a corresponding ear of the user, acoustic transducers 1120(C), 1120(D), 1120(E), 1120(F), 1120(G), and 1120(H), which may be positioned at various locations on frame 1110, and/or acoustic transducers 1120(I) and 1120(J), which may be positioned on a corresponding neckband 115.

In some embodiments, one or more of acoustic transducers 1120(A)-(J) may be used as output transducers (e.g., speakers). For example, acoustic transducers 1120(A) and/or 1120(B) may be earbuds or any other suitable type of headphone or speaker.

The configuration of acoustic transducers 1120 of the microphone array may vary. While augmented-reality system 1100 is shown in FIG. 11 as having ten acoustic transducers 1120, the number of acoustic transducers 1120 may be greater or less than ten. In some embodiments, using higher numbers of acoustic transducers 1120 may increase the amount of audio information collected and/or the sensitivity and accuracy of the audio information. In contrast, using a lower number of acoustic transducers 1120 may decrease the computing power required by an associated controller 1150 to process the collected audio information. In addition, the position of each acoustic transducer 1120 of the microphone array may vary. For example, the position of an acoustic transducer 1120 may include a defined position on the user, a defined coordinate on frame 1110, an orientation associated with each acoustic transducer 1120, or some combination thereof.

Acoustic transducers 1120(A) and 1120(B) may be positioned on different parts of the user's ear, such as behind the pinna, behind the tragus, and/or within the auricle or fossa. Or, there may be additional acoustic transducers 1120 on or surrounding the ear in addition to acoustic transducers 1120 inside the ear canal. Having an acoustic transducer 1120 positioned next to an ear canal of a user may enable the microphone array to collect information on how sounds arrive at the ear canal. By positioning at least two of acoustic transducers 1120 on either side of a user's head (e.g., as binaural microphones), augmented-reality device 1100 may simulate binaural hearing and capture a 3D stereo sound field around about a user's head. In some embodiments, acoustic transducers 1120(A) and 1120(B) may be connected to augmented-reality system 1100 via a wired connection 1130, and in other embodiments acoustic transducers 1120(A) and 1120(B) may be connected to augmented-reality system 1100 via a wireless connection (e.g., a BLUETOOTH connection). In still other embodiments, acoustic transducers 1120(A) and 1120(B) may not be used at all in conjunction with augmented-reality system 1100.

Acoustic transducers 1120 on frame 1110 may be positioned in a variety of different ways, including along the length of the temples, across the bridge, above or below display devices 1115(A) and 1115(B), or some combination thereof. Acoustic transducers 1120 may also be oriented such that the microphone array is able to detect sounds in a wide range of directions surrounding the user wearing the augmented-reality system 1100. In some embodiments, an optimization process may be performed during manufacturing of augmented-reality system 1100 to determine relative positioning of each acoustic transducer 1120 in the microphone array.

In some examples, augmented-reality system 1100 may include or be connected to an external device (e.g., a paired device), such as neckband 115. Neckband 115 generally represents any type or form of paired device. Thus, the following discussion of neckband 115 may also apply to various other paired devices, such as charging cases, smart watches, smart phones, wrist bands, other wearable devices, hand-held controllers, tablet computers, laptop computers, other external compute devices, etc.

As shown, neckband 115 may be coupled to eyewear device 1102 via one or more connectors. The connectors may be wired or wireless and may include electrical and/or non-electrical (e.g., structural) components. In some cases, eyewear device 1102 and neckband 115 may operate independently without any wired or wireless connection between them. While FIG. 11 illustrates the components of eyewear device 1102 and neckband 115 in example locations on eyewear device 1102 and neckband 115, the components may be located elsewhere and/or distributed differently on eyewear device 1102 and/or neckband 115. In some embodiments, the components of eyewear device 1102 and neckband 115 may be located on one or more additional peripheral devices paired with eyewear device 1102, neckband 115, or some combination thereof.

Pairing external devices, such as neckband 115, with augmented-reality eyewear devices may enable the eyewear devices to achieve the form factor of a pair of glasses while still providing sufficient battery and computation power for expanded capabilities. Some or all of the battery power, computational resources, and/or additional features of augmented-reality system 1100 may be provided by a paired device or shared between a paired device and an eyewear device, thus reducing the weight, heat profile, and form factor of the eyewear device overall while still retaining desired functionality. For example, neckband 115 may allow components that would otherwise be included on an eyewear device to be included in neckband 115 since users may tolerate a heavier weight load on their shoulders than they would tolerate on their heads. Neckband 115 may also have a larger surface area over which to diffuse and disperse heat to the ambient environment. Thus, neckband 115 may allow for greater battery and computation capacity than might otherwise have been possible on a stand-alone eyewear device. Since weight carried in neckband 115 may be less invasive to a user than weight carried in eyewear device 1102, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

Neckband 115 may be communicatively coupled with eyewear device 1102 and/or to other devices. These other devices may provide certain functions (e.g., tracking, localizing, depth mapping, processing, storage, etc.) to augmented-reality system 1100. In the embodiment of FIG. 11, neckband 115 may include two acoustic transducers (e.g., 1120(I) and 1120(J)) that are part of the microphone array (or potentially form their own microphone subarray). Neckband 115 may also include a controller 1125 and a power source 1135.

Acoustic transducers 1120(I) and 1120(J) of neckband 115 may be configured to detect sound and convert the detected sound into an electronic format (analog or digital). In the embodiment of FIG. 11, acoustic transducers 1120(I) and 1120(J) may be positioned on neckband 115, thereby increasing the distance between the neckband acoustic transducers 1120(I) and 1120(J) and other acoustic transducers 1120 positioned on eyewear device 1102. In some cases, increasing the distance between acoustic transducers 1120 of the microphone array may improve the accuracy of beamforming performed via the microphone array. For example, if a sound is detected by acoustic transducers 1120(C) and 1120(D) and the distance between acoustic transducers 1120(C) and 1120(D) is greater than, e.g., the distance between acoustic transducers 1120(D) and 1120(E), the determined source location of the detected sound may be more accurate than if the sound had been detected by acoustic transducers 1120(D) and 1120(E).

Controller 1125 of neckband 115 may process information generated by the sensors on neckband 115 and/or augmented-reality system 1100. For example, controller 1125 may process information from the microphone array that describes sounds detected by the microphone array. For each detected sound, controller 1125 may perform a direction-of-arrival (DOA) estimation to estimate a direction from which the detected sound arrived at the microphone array. As the microphone array detects sounds, controller 1125 may populate an audio data set with the information. In embodiments in which augmented-reality system 1100 includes an inertial measurement unit, controller 1125 may compute all inertial and spatial calculations from the IMU located on eyewear device 1102. A connector may convey information between augmented-reality system 1100 and neckband 115 and between augmented-reality system 1100 and controller 1125. The information may be in the form of optical data, electrical data, wireless data, or any other transmittable data form. Moving the processing of information generated by augmented-reality system 1100 to neckband 115 may reduce weight and heat in eyewear device 1102, making it more comfortable to the user.

Power source 1135 in neckband 115 may provide power to eyewear device 1102 and/or to neckband 115. Power source 1135 may include, without limitation, lithium ion batteries, lithium-polymer batteries, primary lithium batteries, alkaline batteries, or any other form of power storage. In some cases, power source 1135 may be a wired power source. Including power source 1135 on neckband 115 instead of on eyewear device 1102 may help better distribute the weight and heat generated by power source 1135.

As noted, some artificial-reality systems may, instead of blending an artificial reality with actual reality, substantially replace one or more of a user's sensory perceptions of the real world with a virtual experience. One example of this type of system is a head-worn display system, such as virtual-reality system 1200 in FIG. 12, that mostly or completely covers a user's field of view. Virtual-reality system 1200 may include a front rigid body 1202 and a band 124 shaped to fit around a user's head. Virtual-reality system 1200 may also include output audio transducers 126(A) and 126(B). Furthermore, while not shown in FIG. 12, front rigid body 1202 may include one or more electronic elements, including one or more electronic displays, one or more inertial measurement units (IMUS), one or more tracking emitters or detectors, and/or any other suitable device or system for creating an artificial-reality experience.

Artificial-reality systems may include a variety of types of visual feedback mechanisms. For example, display devices in augmented-reality system 1100 and/or virtual-reality system 1200 may include one or more liquid crystal displays (LCDs), light emitting diode (LED) displays, microLED displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in augmented-reality system 1100 and/or virtual-reality system 1200 may include micro-LED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, augmented-reality system 1100 and/or virtual-reality system 1200 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

Some augmented-reality systems may map a user's and/or device's environment using techniques referred to as “simultaneous location and mapping” (SLAM). SLAM mapping and location identifying techniques may involve a variety of hardware and software tools that can create or update a map of an environment while simultaneously keeping track of a user's location within the mapped environment. SLAM may use many different types of sensors to create a map and determine a user's position within the map.

SLAM techniques may, for example, implement optical sensors to determine a user's location. Radios including WiFi, BLUETOOTH, global positioning system (GPS), cellular or other communication devices may be also used to determine a user's location relative to a radio transceiver or group of transceivers (e.g., a WiFi router or group of GPS satellites). Acoustic sensors such as microphone arrays or 2D or 3D sonar sensors may also be used to determine a user's location within an environment. Augmented-reality and virtual-reality devices (such as systems 1100 and 1200 of FIGS. 11 and 12, respectively) may incorporate any or all of these types of sensors to perform SLAM operations such as creating and continually updating maps of the user's current environment. In at least some of the embodiments described herein, SLAM data generated by these sensors may be referred to as “environmental data” and may indicate a user's current environment. This data may be stored in a local or remote data store (e.g., a cloud data store) and may be provided to a user's AR/VR device on demand.

As noted, artificial-reality systems 1100 and 1200 may be used with a variety of other types of devices to provide a more compelling artificial-reality experience. These devices may be haptic interfaces with transducers that provide haptic feedback and/or that collect haptic information about a user's interaction with an environment. The artificial-reality systems disclosed herein may include various types of haptic interfaces that detect or convey various types of haptic information, including tactile feedback (e.g., feedback that a user detects via nerves in the skin, which may also be referred to as cutaneous feedback) and/or kinesthetic feedback (e.g., feedback that a user detects via receptors located in muscles, joints, and/or tendons).

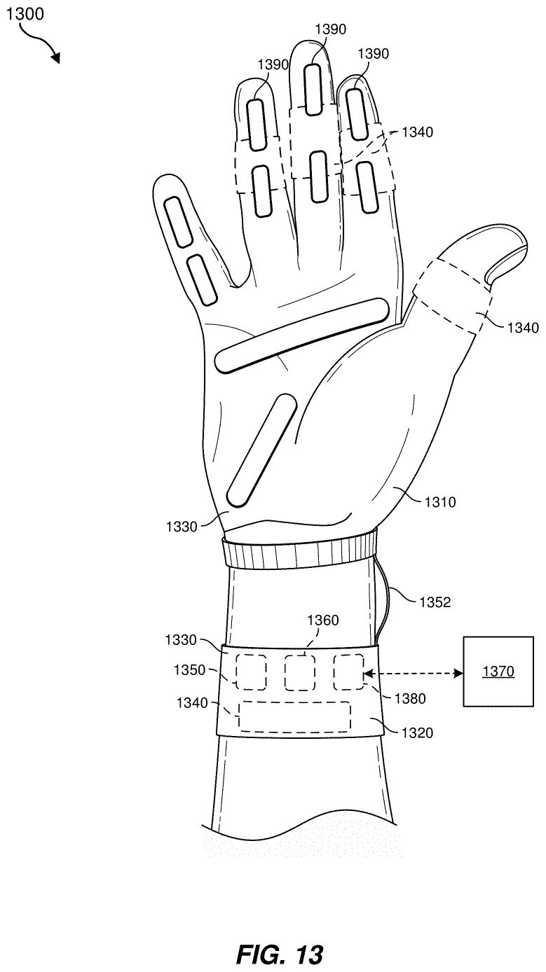

Haptic feedback may be provided by interfaces positioned within a user's environment (e.g., chairs, tables, floors, etc.) and/or interfaces on articles that may be worn or carried by a user (e.g., gloves, wristbands, etc.). As an example, FIG. 13 illustrates a vibrotactile system 1300 in the form of a wearable glove (haptic device 1310) and wristband (haptic device 1320). Haptic device 1310 and haptic device 1320 are shown as examples of wearable devices that include a flexible, wearable textile material 1330 that is shaped and configured for positioning against a user's hand and wrist, respectively. This disclosure also includes vibrotactile systems that may be shaped and configured for positioning against other human body parts, such as a finger, an arm, a head, a torso, a foot, or a leg. By way of example and not limitation, vibrotactile systems according to various embodiments of the present disclosure may also be in the form of a glove, a headband, an armband, a sleeve, a head covering, a sock, a shirt, or pants, among other possibilities. In some examples, the term “textile” may include any flexible, wearable material, including woven fabric, non-woven fabric, leather, cloth, a flexible polymer material, composite materials, etc.

One or more vibrotactile devices 1340 may be positioned at least partially within one or more corresponding pockets formed in textile material 1330 of vibrotactile system 1300. Vibrotactile devices 1340 may be positioned in locations to provide a vibrating sensation (e.g., haptic feedback) to a user of vibrotactile system 1300. For example, vibrotactile devices 1340 may be positioned against the user's finger(s), thumb, or wrist, as shown in FIG. 13. Vibrotactile devices 1340 may, in some examples, be sufficiently flexible to conform to or bend with the user's corresponding body part(s).

A power source 1350 (e.g., a battery) for applying a voltage to the vibrotactile devices 1340 for activation thereof may be electrically coupled to vibrotactile devices 1340, such as via conductive wiring 1352. In some examples, each of vibrotactile devices 1340 may be independently electrically coupled to power source 1350 for individual activation. In some embodiments, a processor 1360 may be operatively coupled to power source 1350 and configured (e.g., programmed) to control activation of vibrotactile devices 1340.

Vibrotactile system 1300 may be implemented in a variety of ways. In some examples, vibrotactile system 1300 may be a standalone system with integral subsystems and components for operation independent of other devices and systems. As another example, vibrotactile system 1300 may be configured for interaction with another device or system 1370. For example, vibrotactile system 1300 may, in some examples, include a communications interface 1380 for receiving and/or sending signals to the other device or system 1370. The other device or system 1370 may be a mobile device, a gaming console, an artificial-reality (e.g., virtual-reality, augmented-reality, mixed-reality) device, a personal computer, a tablet computer, a network device (e.g., a modem, a router, etc.), a handheld controller, etc. Communications interface 1380 may enable communications between vibrotactile system 1300 and the other device or system 1370 via a wireless (e.g., Wi-Fi, BLUETOOTH, cellular, radio, etc.) link or a wired link. If present, communications interface 1380 may be in communication with processor 1360, such as to provide a signal to processor 1360 to activate or deactivate one or more of the vibrotactile devices 1340.

Vibrotactile system 1300 may optionally include other subsystems and components, such as touch-sensitive pads 1390, pressure sensors, motion sensors, position sensors, lighting elements, and/or user interface elements (e.g., an on/off button, a vibration control element, etc.). During use, vibrotactile devices 1340 may be configured to be activated for a variety of different reasons, such as in response to the user's interaction with user interface elements, a signal from the motion or position sensors, a signal from the touch-sensitive pads 1390, a signal from the pressure sensors, a signal from the other device or system 1370, etc.

Although power source 1350, processor 1360, and communications interface 1380 are illustrated in FIG. 13 as being positioned in haptic device 1320, the present disclosure is not so limited. For example, one or more of power source 1350, processor 1360, or communications interface 1380 may be positioned within haptic device 1310 or within another wearable textile.

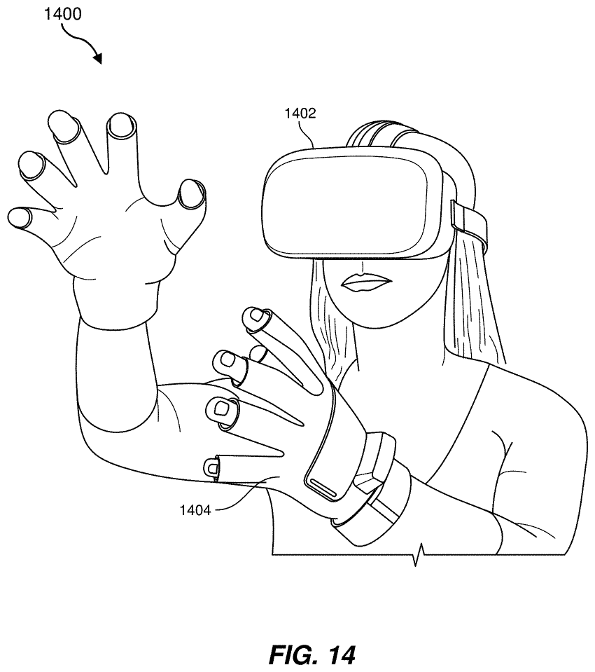

Haptic wearables, such as those shown in and described in connection with FIG. 13, may be implemented in a variety of types of artificial-reality systems and environments. FIG. 14 shows an example artificial-reality environment 1400 including one head-mounted virtual-reality display and two haptic devices (i.e., gloves), and in other embodiments any number and/or combination of these components and other components may be included in an artificial-reality system. For example, in some embodiments there may be multiple head-mounted displays each having an associated haptic device, with each head-mounted display and each haptic device communicating with the same console, portable computing device, or other computing system.

Head-mounted display 1402 generally represents any type or form of virtual-reality system, such as virtual-reality system 1200 in FIG. 12. Haptic device 144 generally represents any type or form of wearable device, worn by a user of an artificial-reality system, that provides haptic feedback to the user to give the user the perception that he or she is physically engaging with a virtual object. In some embodiments, haptic device 144 may provide haptic feedback by applying vibration, motion, and/or force to the user. For example, haptic device 144 may limit or augment a user's movement. To give a specific example, haptic device 144 may limit a user's hand from moving forward so that the user has the perception that his or her hand has come in physical contact with a virtual wall. In this specific example, one or more actuators within the haptic device may achieve the physical-movement restriction by pumping fluid into an inflatable bladder of the haptic device. In some examples, a user may also use haptic device 144 to send action requests to a console. Examples of action requests include, without limitation, requests to start an application and/or end the application and/or requests to perform a particular action within the application.

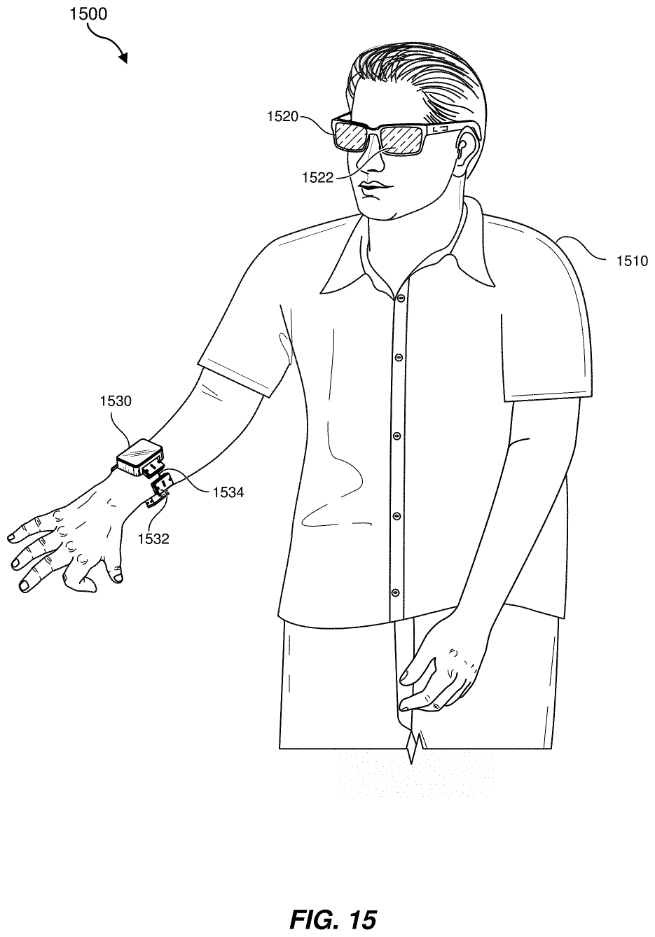

While haptic interfaces may be used with virtual-reality systems, as shown in FIG. 14, haptic interfaces may also be used with augmented-reality systems, as shown in FIG. 15. FIG. 15 is a perspective view of a user 1510 interacting with an augmented-reality system 1500. In this example, user 1510 may wear a pair of augmented-reality glasses 1520 that may have one or more displays 1522 and that are paired with a haptic device 1530. In this example, haptic device 1530 may be a wristband that includes a plurality of band elements 1532 and a tensioning mechanism 1534 that connects band elements 1532 to one another.

One or more of band elements 1532 may include any type or form of actuator suitable for providing haptic feedback. For example, one or more of band elements 1532 may be configured to provide one or more of various types of cutaneous feedback, including vibration, force, traction, texture, and/or temperature. To provide such feedback, band elements 1532 may include one or more of various types of actuators. In one example, each of band elements 1532 may include a vibrotactor (e.g., a vibrotactile actuator) configured to vibrate in unison or independently to provide one or more of various types of haptic sensations to a user. Alternatively, only a single band element or a subset of band elements may include vibrotactors.

Haptic devices 1310, 1320, 144, and 1530 may include any suitable number and/or type of haptic transducer, sensor, and/or feedback mechanism. For example, haptic devices 1310, 1320, 144, and 1530 may include one or more mechanical transducers, piezoelectric transducers, and/or fluidic transducers. Haptic devices 1310, 1320, 144, and 1530 may also include various combinations of different types and forms of transducers that work together or independently to enhance a user's artificial-reality experience. In one example, each of band elements 1532 of haptic device 1530 may include a vibrotactor (e.g., a vibrotactile actuator) configured to vibrate in unison or independently to provide one or more of various types of haptic sensations to a user.

In some embodiments, the systems described herein may also include an eye-tracking subsystem designed to identify and track various characteristics of a user's eye(s), such as the user's gaze direction. The phrase “eye tracking” may, in some examples, refer to a process by which the position, orientation, and/or motion of an eye is measured, detected, sensed, determined, and/or monitored. The disclosed systems may measure the position, orientation, and/or motion of an eye in a variety of different ways, including through the use of various optical-based eye-tracking techniques, ultrasound-based eye-tracking techniques, etc. An eye-tracking subsystem may be configured in a number of different ways and may include a variety of different eye-tracking hardware components or other computer-vision components. For example, an eye-tracking subsystem may include a variety of different optical sensors, such as two-dimensional (2D) or 3D cameras, time-of-flight depth sensors, single-beam or sweeping laser rangefinders, 3D LiDAR sensors, and/or any other suitable type or form of optical sensor. In this example, a processing subsystem may process data from one or more of these sensors to measure, detect, determine, and/or otherwise monitor the position, orientation, and/or motion of the user's eye(s).

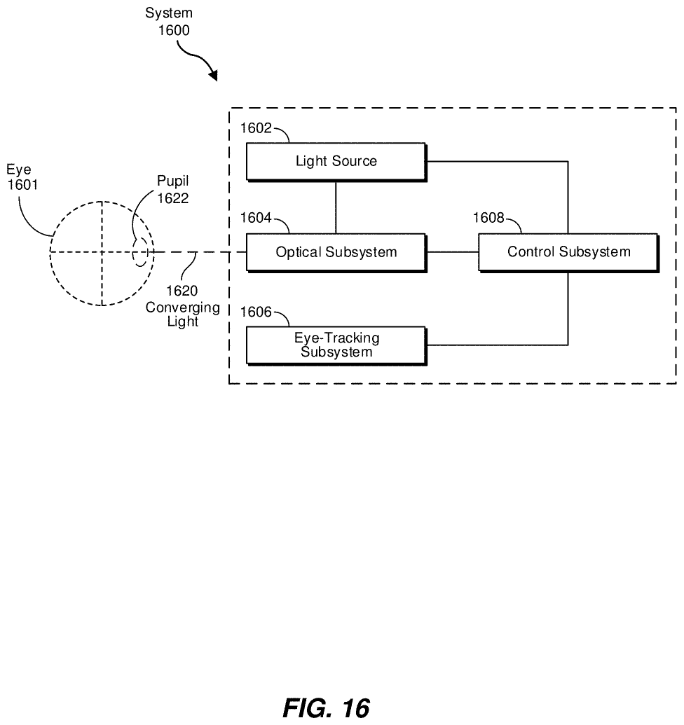

FIG. 16 is an illustration of an exemplary system 1600 that incorporates an eye-tracking subsystem capable of tracking a user's eye(s). As depicted in FIG. 16, system 1600 may include a light source 1602, an optical subsystem 164, an eye-tracking subsystem 166, and/or a control subsystem 168. In some examples, light source 1602 may generate light for an image (e.g., to be presented to an eye 1601 of the viewer). Light source 1602 may represent any of a variety of suitable devices. For example, light source 1602 can include a two-dimensional projector (e.g., a LCoS display), a scanning source (e.g., a scanning laser), or other device (e.g., an LCD, an LED display, an OLED display, an active-matrix OLED display (AMOLED), a transparent OLED display (TOLED), a waveguide, or some other display capable of generating light for presenting an image to the viewer). In some examples, the image may represent a virtual image, which may refer to an optical image formed from the apparent divergence of light rays from a point in space, as opposed to an image formed from the light ray's actual divergence.

In some embodiments, optical subsystem 164 may receive the light generated by light source 1602 and generate, based on the received light, converging light 1620 that includes the image. In some examples, optical subsystem 164 may include any number of lenses (e.g., Fresnel lenses, convex lenses, concave lenses), apertures, filters, mirrors, prisms, and/or other optical components, possibly in combination with actuators and/or other devices. In particular, the actuators and/or other devices may translate and/or rotate one or more of the optical components to alter one or more aspects of converging light 1620. Further, various mechanical couplings may serve to maintain the relative spacing and/or the orientation of the optical components in any suitable combination.

In one embodiment, eye-tracking subsystem 166 may generate tracking information indicating a gaze angle of an eye 1601 of the viewer. In this embodiment, control subsystem 168 may control aspects of optical subsystem 164 (e.g., the angle of incidence of converging light 1620) based at least in part on this tracking information. Additionally, in some examples, control subsystem 168 may store and utilize historical tracking information (e.g., a history of the tracking information over a given duration, such as the previous second or fraction thereof) to anticipate the gaze angle of eye 1601 (e.g., an angle between the visual axis and the anatomical axis of eye 1601). In some embodiments, eye-tracking subsystem 166 may detect radiation emanating from some portion of eye 1601 (e.g., the cornea, the iris, the pupil, or the like) to determine the current gaze angle of eye 1601. In other examples, eye-tracking subsystem 166 may employ a wavefront sensor to track the current location of the pupil.

Any number of techniques can be used to track eye 1601. Some techniques may involve illuminating eye 1601 with infrared light and measuring reflections with at least one optical sensor that is tuned to be sensitive to the infrared light. Information about how the infrared light is reflected from eye 1601 may be analyzed to determine the position(s), orientation(s), and/or motion(s) of one or more eye feature(s), such as the cornea, pupil, iris, and/or retinal blood vessels.

In some examples, the radiation captured by a sensor of eye-tracking subsystem 166 may be digitized (i.e., converted to an electronic signal). Further, the sensor may transmit a digital representation of this electronic signal to one or more processors (for example, processors associated with a device including eye-tracking subsystem 166). Eye-tracking subsystem 166 may include any of a variety of sensors in a variety of different configurations. For example, eye-tracking subsystem 166 may include an infrared detector that reacts to infrared radiation. The infrared detector may be a thermal detector, a photonic detector, and/or any other suitable type of detector. Thermal detectors may include detectors that react to thermal effects of the incident infrared radiation.

In some examples, one or more processors may process the digital representation generated by the sensor(s) of eye-tracking subsystem 166 to track the movement of eye 1601. In another example, these processors may track the movements of eye 1601 by executing algorithms represented by computer-executable instructions stored on non-transitory memory. In some examples, on-chip logic (e.g., an application-specific integrated circuit or ASIC) may be used to perform at least portions of such algorithms. As noted, eye-tracking subsystem 166 may be programmed to use an output of the sensor(s) to track movement of eye 1601. In some embodiments, eye-tracking subsystem 166 may analyze the digital representation generated by the sensors to extract eye rotation information from changes in reflections. In one embodiment, eye-tracking subsystem 166 may use corneal reflections or glints (also known as Purkinje images) and/or the center of the eye's pupil 1622 as features to track over time.

In some embodiments, eye-tracking subsystem 166 may use the center of the eye's pupil 1622 and infrared or near-infrared, non-collimated light to create corneal reflections. In these embodiments, eye-tracking subsystem 166 may use the vector between the center of the eye's pupil 1622 and the corneal reflections to compute the gaze direction of eye 1601. In some embodiments, the disclosed systems may perform a calibration procedure for an individual (using, e.g., supervised or unsupervised techniques) before tracking the user's eyes. For example, the calibration procedure may include directing users to look at one or more points displayed on a display while the eye-tracking system records the values that correspond to each gaze position associated with each point.

In some embodiments, eye-tracking subsystem 166 may use two types of infrared and/or near-infrared (also known as active light) eye-tracking techniques: bright-pupil and dark-pupil eye tracking, which may be differentiated based on the location of an illumination source with respect to the optical elements used. If the illumination is coaxial with the optical path, then eye 1601 may act as a retroreflector as the light reflects off the retina, thereby creating a bright pupil effect similar to a red-eye effect in photography. If the illumination source is offset from the optical path, then the eye's pupil 1622 may appear dark because the retroreflection from the retina is directed away from the sensor. In some embodiments, bright-pupil tracking may create greater iris/pupil contrast, allowing more robust eye tracking with iris pigmentation, and may feature reduced interference (e.g., interference caused by eyelashes and other obscuring features). Bright-pupil tracking may also allow tracking in lighting conditions ranging from total darkness to a very bright environment.

In some embodiments, control subsystem 168 may control light source 1602 and/or optical subsystem 164 to reduce optical aberrations (e.g., chromatic aberrations and/or monochromatic aberrations) of the image that may be caused by or influenced by eye 1601. In some examples, as mentioned above, control subsystem 168 may use the tracking information from eye-tracking subsystem 166 to perform such control. For example, in controlling light source 1602, control subsystem 168 may alter the light generated by light source 1602 (e.g., by way of image rendering) to modify (e.g., pre-distort) the image so that the aberration of the image caused by eye 1601 is reduced.

The disclosed systems may track both the position and relative size of the pupil (since, e.g., the pupil dilates and/or contracts). In some examples, the eye-tracking devices and components (e.g., sensors and/or sources) used for detecting and/or tracking the pupil may be different (or calibrated differently) for different types of eyes. For example, the frequency range of the sensors may be different (or separately calibrated) for eyes of different colors and/or different pupil types, sizes, and/or the like. As such, the various eye-tracking components (e.g., infrared sources and/or sensors) described herein may need to be calibrated for each individual user and/or eye.

The disclosed systems may track both eyes with and without ophthalmic correction, such as that provided by contact lenses worn by the user. In some embodiments, ophthalmic correction elements (e.g., adjustable lenses) may be directly incorporated into the artificial reality systems described herein. In some examples, the color of the user's eye may necessitate modification of a corresponding eye-tracking algorithm. For example, eye-tracking algorithms may need to be modified based at least in part on the differing color contrast between a brown eye and, for example, a blue eye.

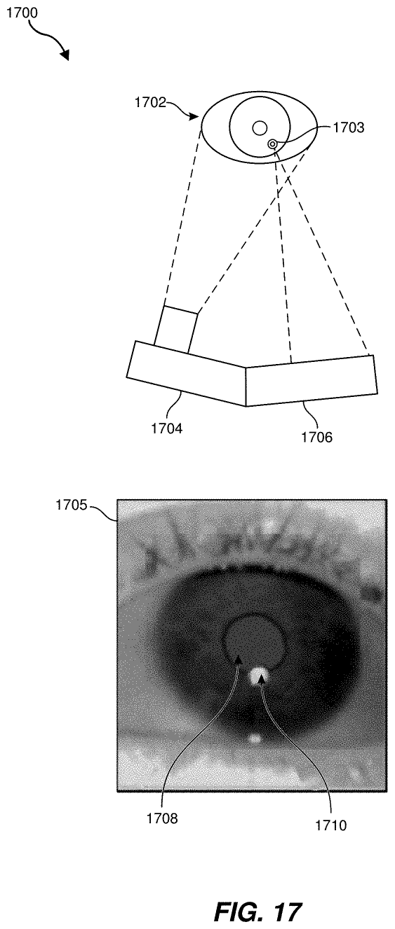

FIG. 17 is a more detailed illustration of various aspects of the eye-tracking subsystem illustrated in FIG. 16. As shown in this figure, an eye-tracking subsystem 1700 may include at least one source 174 and at least one sensor 176. Source 174 generally represents any type or form of element capable of emitting radiation. In one example, source 174 may generate visible, infrared, and/or near-infrared radiation. In some examples, source 174 may radiate non-collimated infrared and/or near-infrared portions of the electromagnetic spectrum towards an eye 1702 of a user. Source 174 may utilize a variety of sampling rates and speeds. For example, the disclosed systems may use sources with higher sampling rates in order to capture fixational eye movements of a user's eye 1702 and/or to correctly measure saccade dynamics of the user's eye 1702. As noted above, any type or form of eye-tracking technique may be used to track the user's eye 1702, including optical-based eye-tracking techniques, ultrasound-based eye-tracking techniques, etc.

Sensor 176 generally represents any type or form of element capable of detecting radiation, such as radiation reflected off the user's eye 1702. Examples of sensor 176 include, without limitation, a charge coupled device (CCD), a photodiode array, a complementary metal-oxide-semiconductor (CMOS) based sensor device, and/or the like. In one example, sensor 176 may represent a sensor having predetermined parameters, including, but not limited to, a dynamic resolution range, linearity, and/or other characteristic selected and/or designed specifically for eye tracking.

As detailed above, eye-tracking subsystem 1700 may generate one or more glints. As detailed above, a glint 173 may represent reflections of radiation (e.g., infrared radiation from an infrared source, such as source 174) from the structure of the user's eye. In various embodiments, glint 173 and/or the user's pupil may be tracked using an eye-tracking algorithm executed by a processor (either within or external to an artificial reality device). For example, an artificial reality device may include a processor and/or a memory device in order to perform eye tracking locally and/or a transceiver to send and receive the data necessary to perform eye tracking on an external device (e.g., a mobile phone, cloud server, or other computing device).

FIG. 17 shows an example image 175 captured by an eye-tracking subsystem, such as eye-tracking subsystem 1700. In this example, image 175 may include both the user's pupil 178 and a glint 1710 near the same. In some examples, pupil 178 and/or glint 1710 may be identified using an artificial-intelligence-based algorithm, such as a computer-vision-based algorithm. In one embodiment, image 175 may represent a single frame in a series of frames that may be analyzed continuously in order to track the eye 1702 of the user. Further, pupil 178 and/or glint 1710 may be tracked over a period of time to determine a user's gaze.

In one example, eye-tracking subsystem 1700 may be configured to identify and measure the inter-pupillary distance (IPD) of a user. In some embodiments, eye-tracking subsystem 1700 may measure and/or calculate the IPD of the user while the user is wearing the artificial reality system. In these embodiments, eye-tracking subsystem 1700 may detect the positions of a user's eyes and may use this information to calculate the user's IPD.

As noted, the eye-tracking systems or subsystems disclosed herein may track a user's eye position and/or eye movement in a variety of ways. In one example, one or more light sources and/or optical sensors may capture an image of the user's eyes. The eye-tracking subsystem may then use the captured information to determine the user's inter-pupillary distance, interocular distance, and/or a 3D position of each eye (e.g., for distortion adjustment purposes), including a magnitude of torsion and rotation (i.e., roll, pitch, and yaw) and/or gaze directions for each eye. In one example, infrared light may be emitted by the eye-tracking subsystem and reflected from each eye. The reflected light may be received or detected by an optical sensor and analyzed to extract eye rotation data from changes in the infrared light reflected by each eye.

The eye-tracking subsystem may use any of a variety of different methods to track the eyes of a user. For example, a light source (e.g., infrared light-emitting diodes) may emit a dot pattern onto each eye of the user. The eye-tracking subsystem may then detect (e.g., via an optical sensor coupled to the artificial reality system) and analyze a reflection of the dot pattern from each eye of the user to identify a location of each pupil of the user. Accordingly, the eye-tracking subsystem may track up to six degrees of freedom of each eye (i.e., 3D position, roll, pitch, and yaw) and at least a subset of the tracked quantities may be combined from two eyes of a user to estimate a gaze point (i.e., a 3D location or position in a virtual scene where the user is looking) and/or an IPD.

In some cases, the distance between a user's pupil and a display may change as the user's eye moves to look in different directions. The varying distance between a pupil and a display as viewing direction changes may be referred to as “pupil swim” and may contribute to distortion perceived by the user as a result of light focusing in different locations as the distance between the pupil and the display changes. Accordingly, measuring distortion at different eye positions and pupil distances relative to displays and generating distortion corrections for different positions and distances may allow mitigation of distortion caused by pupil swim by tracking the 3D position of a user's eyes and applying a distortion correction corresponding to the 3D position of each of the user's eyes at a given point in time. Thus, knowing the 3D position of each of a user's eyes may allow for the mitigation of distortion caused by changes in the distance between the pupil of the eye and the display by applying a distortion correction for each 3D eye position. Furthermore, as noted above, knowing the position of each of the user's eyes may also enable the eye-tracking subsystem to make automated adjustments for a user's IPD.

In some embodiments, a display subsystem may include a variety of additional subsystems that may work in conjunction with the eye-tracking subsystems described herein. For example, a display subsystem may include a varifocal subsystem, a scene-rendering module, and/or a vergence-processing module. The varifocal subsystem may cause left and right display elements to vary the focal distance of the display device. In one embodiment, the varifocal subsystem may physically change the distance between a display and the optics through which it is viewed by moving the display, the optics, or both. Additionally, moving or translating two lenses relative to each other may also be used to change the focal distance of the display. Thus, the varifocal subsystem may include actuators or motors that move displays and/or optics to change the distance between them. This varifocal subsystem may be separate from or integrated into the display subsystem. The varifocal subsystem may also be integrated into or separate from its actuation subsystem and/or the eye-tracking subsystems described herein.

In one example, the display subsystem may include a vergence-processing module configured to determine a vergence depth of a user's gaze based on a gaze point and/or an estimated intersection of the gaze lines determined by the eye-tracking subsystem. Vergence may refer to the simultaneous movement or rotation of both eyes in opposite directions to maintain single binocular vision, which may be naturally and automatically performed by the human eye. Thus, a location where a user's eyes are verged is where the user is looking and is also typically the location where the user's eyes are focused. For example, the vergence-processing module may triangulate gaze lines to estimate a distance or depth from the user associated with intersection of the gaze lines. The depth associated with intersection of the gaze lines may then be used as an approximation for the accommodation distance, which may identify a distance from the user where the user's eyes are directed. Thus, the vergence distance may allow for the determination of a location where the user's eyes should be focused and a depth from the user's eyes at which the eyes are focused, thereby providing information (such as an object or plane of focus) for rendering adjustments to the virtual scene.

The vergence-processing module may coordinate with the eye-tracking subsystems described herein to make adjustments to the display subsystem to account for a user's vergence depth. When the user is focused on something at a distance, the user's pupils may be slightly farther apart than when the user is focused on something close. The eye-tracking subsystem may obtain information about the user's vergence or focus depth and may adjust the display subsystem to be closer together when the user's eyes focus or verge on something close and to be farther apart when the user's eyes focus or verge on something at a distance.

The eye-tracking information generated by the above-described eye-tracking subsystems may also be used, for example, to modify various aspect of how different computer-generated images are presented. For example, a display subsystem may be configured to modify, based on information generated by an eye-tracking subsystem, at least one aspect of how the computer-generated images are presented. For instance, the computer-generated images may be modified based on the user's eye movement, such that if a user is looking up, the computer-generated images may be moved upward on the screen. Similarly, if the user is looking to the side or down, the computer-generated images may be moved to the side or downward on the screen. If the user's eyes are closed, the computer-generated images may be paused or removed from the display and resumed once the user's eyes are back open.

The above-described eye-tracking subsystems can be incorporated into one or more of the various artificial reality systems described herein in a variety of ways. For example, one or more of the various components of system 1600 and/or eye-tracking subsystem 1700 may be incorporated into augmented-reality system 1100 in FIG. 11 and/or virtual-reality system 1200 in FIG. 12 to enable these systems to perform various eye-tracking tasks (including one or more of the eye-tracking operations described herein).

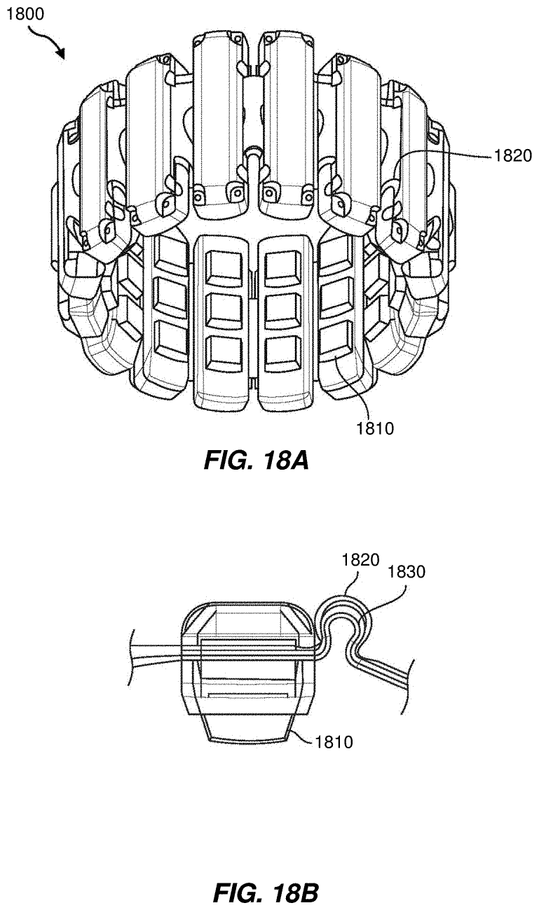

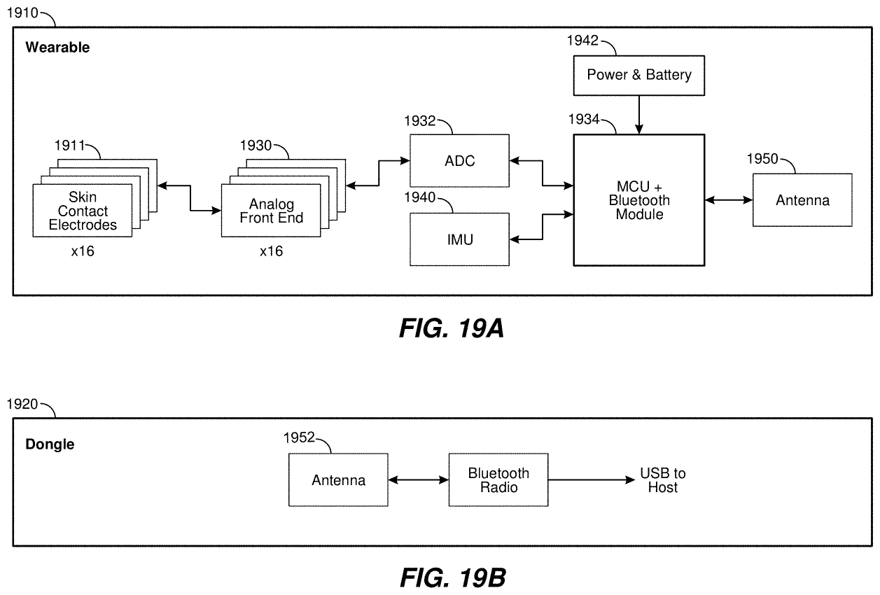

FIG. 18A illustrates an exemplary human-machine interface (also referred to herein as an EMG control interface) configured to be worn around a user's lower arm or wrist as a wearable system 1800. In this example, wearable system 1800 may include sixteen neuromuscular sensors 1810 (e.g., EMG sensors) arranged circumferentially around an elastic band 1820 with an interior surface 1830 configured to contact a user's skin. However, any suitable number of neuromuscular sensors may be used. The number and arrangement of neuromuscular sensors may depend on the particular application for which the wearable device is used. For example, a wearable armband or wristband can be used to generate control information for controlling an augmented reality system, a robot, controlling a vehicle, scrolling through text, controlling a virtual avatar, or any other suitable control task. As shown, the sensors may be coupled together using flexible electronics incorporated into the wireless device. FIG. 18B illustrates a cross-sectional view through one of the sensors of the wearable device shown in FIG. 18A. In some embodiments, the output of one or more of the sensing components can be optionally processed using hardware signal processing circuitry (e.g., to perform amplification, filtering, and/or rectification). In other embodiments, at least some signal processing of the output of the sensing components can be performed in software. Thus, signal processing of signals sampled by the sensors can be performed in hardware, software, or by any suitable combination of hardware and software, as aspects of the technology described herein are not limited in this respect. A non-limiting example of a signal processing chain used to process recorded data from sensors 1810 is discussed in more detail below with reference to FIGS. 19A and 19B.

FIGS. 19A and 19B illustrate an exemplary schematic diagram with internal components of a wearable system with EMG sensors. As shown, the wearable system may include a wearable portion 1910 (FIG. 19A) and a dongle portion 1920 (FIG. 19B) in communication with the wearable portion 1910 (e.g., via BLUETOOTH or another suitable wireless communication technology). As shown in FIG. 19A, the wearable portion 1910 may include skin contact electrodes 1911, examples of which are described in connection with FIGS. 18A and 18B. The output of the skin contact electrodes 1911 may be provided to analog front end 1930, which may be configured to perform analog processing (e.g., amplification, noise reduction, filtering, etc.) on the recorded signals. The processed analog signals may then be provided to analog-to-digital converter 1932, which may convert the analog signals to digital signals that can be processed by one or more computer processors. An example of a computer processor that may be used in accordance with some embodiments is microcontroller (MCU) 1934, illustrated in FIG. 19A. As shown, MCU 1934 may also include inputs from other sensors (e.g., IMU sensor 1940), and power and battery module 1942. The output of the processing performed by MCU 1934 may be provided to antenna 1950 for transmission to dongle portion 1920 shown in FIG. 19B.

Dongle portion 1920 may include antenna 1952, which may be configured to communicate with antenna 1950 included as part of wearable portion 1910. Communication between antennas 1950 and 1952 may occur using any suitable wireless technology and protocol, non-limiting examples of which include radiofrequency signaling and BLUETOOTH. As shown, the signals received by antenna 1952 of dongle portion 1920 may be provided to a host computer for further processing, display, and/or for effecting control of a particular physical or virtual object or objects.

Although the examples provided with reference to FIGS. 18A-18B and FIGS. 19A-19B are discussed in the context of interfaces with EMG sensors, the techniques described herein for reducing electromagnetic interference can also be implemented in wearable interfaces with other types of sensors including, but not limited to, mechanomyography (MMG) sensors, sonomyography (SMG) sensors, and electrical impedance tomography (EIT) sensors. The techniques described herein for reducing electromagnetic interference can also be implemented in wearable interfaces that communicate with computer hosts through wires and cables (e.g., USB cables, optical fiber cables, etc.).

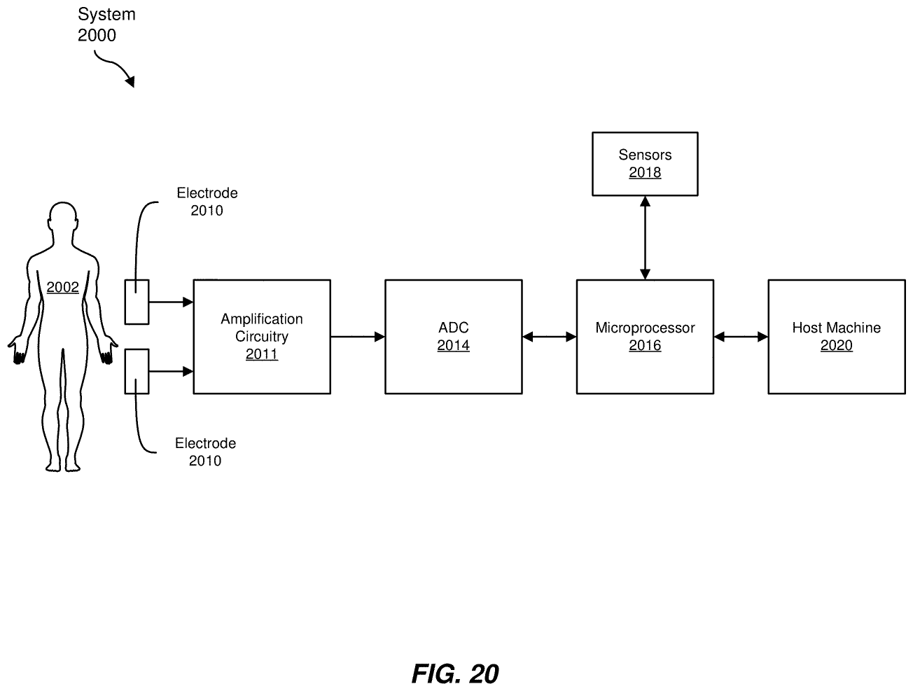

FIG. 20 schematically illustrates components of a biosignal sensing system 2000 in accordance with some embodiments. System 2000 includes a pair of electrodes 2010 (e.g., a pair of dry surface electrodes) configured to register or measure a biosignal (e.g., an Electrooculography (EOG) signal, an Electromyography (EMG) signal, a surface Electromyography (sEMG) signal, an Electroencephalography (EEG) signal, an Electrocardiography (ECG) signal, etc.) generated by the body of a user 2002 (e.g., for electrophysiological monitoring or stimulation). In some embodiments, both of electrodes 2010 may be contact electrodes configured to contact a user's skin. In other embodiments, both of electrodes 2010 may be non-contact electrodes configured to not contact a user's skin. Alternatively, one of electrodes 2010 may be a contact electrode configured to contact a user's skin, and the other one of electrodes 2010 may be a non-contact electrode configured to not contact the user's skin. In some embodiments, electrodes 2010 may be arranged as a portion of a wearable device configured to be worn on or around part of a user's body. For example, in one non-limiting example, a plurality of electrodes including electrodes 2010 may be arranged circumferentially around an adjustable and/or elastic band such as a wristband or armband configured to be worn around a user's wrist or arm (e.g., as illustrated in FIGS. 18A and 18B). Additionally or alternatively, at least some of electrodes 2010 may be arranged on a wearable patch configured to be affixed to or placed in contact with a portion of the body of user 2002. In some embodiments, the electrodes may be minimally invasive and may include one or more conductive components placed in or through all or part of the skin or dermis of the user. It should be appreciated that any suitable number of electrodes may be used, and the number and arrangement of electrodes may depend on the particular application for which a device is used.

Biosignals (e.g., biopotential signals) measured or recorded by electrodes 2010 may be small, and amplification of the biosignals recorded by electrodes 2010 may be desired. As shown in FIG. 20, electrodes 2010 may be coupled to amplification circuitry 2011 configured to amplify the biosignals conducted by electrodes 2010. Amplification circuitry 2011 may include any suitable amplifier. Examples of suitable amplifiers may include operational amplifiers, differential amplifiers that amplify differences between two input voltages, instrumental amplifiers (e.g., differential amplifiers having input buffer amplifiers), single ended amplifiers, and/or any other suitable amplifier capable of amplifying biosignals.

As shown in FIG. 20, an output of amplification circuitry 2011 may be provided to analog-to-digital converter (ADC) circuitry 2014, which may convert amplified biosignals to digital signals for further processing by a microprocessor 2016. In some embodiments, microprocessor 2016 may process the digital signals to enhance remote or virtual social experiences (e.g., by converting or transforming the biosignals into an estimation of a spatial relationship of one or more skeletal structures in the body of user 2002 and/or a force exerted by at least one the skeletal structures in the body of user 2002). Microprocessor 2016 may be implemented by one or more hardware processors. In some embodiments, electrodes 2010, amplification circuitry 2011, ADC circuitry 2014, and/or microprocessor 2016 may represent some or all of a single biosignal sensor. The processed signals output from microprocessor 2016 may be interpreted by a host machine 2020, examples of which include, but are not limited to, a desktop computer, a laptop computer, a smartwatch, a smartphone, a head-mounted display device, or any other computing device. In some implementations, host machine 2020 may be configured to output one or more control signals for controlling a physical or virtual device or object based, at least in part, on an analysis of the signals output from microprocessor 2016. As shown, biosignal sensing system 2000 may include additional sensors 2018, which may be configured to record types of information about a state of a user other than biosignal information. For example, sensors 2018 may include, temperature sensors configured to measure skin/electrode temperature, inertial measurement unit (IMU) sensors configured to measure movement information such as rotation and acceleration, humidity sensors, and other bio-chemical sensors configured to provide information about the user and/or the user's environment.

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive biosignals (e.g., biosignals containing eye-tracking data) to be transformed, transform the biosignals into a prediction of a transition to or from a cognitive state of the user, output a result of the transformation to an intelligent-facilitation subsystem, and/or use the result of the transformation to perform one or more assistive actions and/or interventions that reduce cognitive loads associated with the cognitive state. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A computer-implemented method comprising:
acquiring, via one or more biosensors, one or more biosignals generated by a user of a computing system, the computing system comprising an intelligent-facilitation subsystem adapted to perform one or more assistive actions to reduce the user's cognitive load;
using the one or more biosignals to anticipate a transition to or from a cognitive state of the user; and
providing, to the intelligent-facilitation subsystem, a signal indicating the transition to or from the cognitive state of the user.

2. The computer-implemented method of claim 1, wherein the acquiring, the using, and the providing are performed when the user is not attentively engaged with the computing system.

3. The computer-implemented method of claim 1, wherein:
the one or more biosensors comprise one or more eye-tracking sensors;
the one or more biosignals comprise signals indicative of gaze dynamics of the user; and
the signals indicative of gaze dynamics of the user are used to anticipate the transition to or from the cognitive state of the user.

4. The computer-implemented method of claim 3, wherein the signals indicative of gaze dynamics of the user comprise a measure of gaze velocity.

5. The computer-implemented method of claim 3, wherein the signals indicative of gaze dynamics of the user comprise at least one of:
a measure of ambient attention; or
a measure of focal attention.

6. The computer-implemented method of claim 3, wherein the signals indicative of gaze dynamics of the user comprise a measure of saccade dynamics.

7. The computer-implemented method of claim 1, wherein the cognitive state of the user comprises a state of encoding information to working memory of the user.

8. The computer-implemented method of claim 1, wherein the cognitive state of the user comprises a state of visual searching.

9. The computer-implemented method of claim 1, wherein the cognitive state of the user comprises a state of storing information to long-term memory of the user.

10. The computer-implemented method of claim 1, wherein the cognitive state of the user comprises a state of retrieving information from long-term memory of the user.

11. The computer-implemented method of claim 1, further comprising:
receiving, by the intelligent-facilitation subsystem, the signal indicating the transition to or from the cognitive state of the user; and
performing, by the intelligent-facilitation subsystem, the one or more assistive actions to reduce the user's cognitive load.

12. The computer-implemented method of claim 11, wherein:
using the one or more biosignals to anticipate the transition to or from the cognitive state of the user comprises using the one or more biosignals to anticipate the user's intent to encode information into working memory of the user; and
performing the one or more assistive actions to reduce the user's cognitive load comprises:
presenting, to the user, at least one of:
a virtual notepad;
a virtual list; or
a virtual sketchpad;
receiving, from the user, input indicative of the information; and
storing, by the intelligent-facilitation subsystem, a representation of the information for later retrieval and presentation to the user.

13. The computer-implemented method of claim 11, wherein:
the computing system comprises physical memory; and
performing the one or more assistive actions to reduce the user's cognitive load comprises:
identifying, by the intelligent-facilitation subsystem, at least one attribute of the user's environment that is likely to be encoded into working memory of the user; and
storing the attribute to the physical memory for later retrieval and presentation to the user.

14. The computer-implemented method of claim 13, wherein the intelligent-facilitation subsystem refrains from identifying the at least one attribute of the user's environment until after receiving the signal indicating the transition to or from the cognitive state of the user.

15. A system comprising:
an intelligent-facilitation subsystem adapted to perform one or more assistive actions to reduce a user's cognitive load;
one or more biosensors adapted to detect biosignals generated by the user;
at least one physical processor; and
physical memory comprising computer-executable instructions that, when executed by the physical processor, cause the physical processor to:
acquire, via the one or more biosensors, one or more biosignals generated by the user;
use the one or more biosignals to anticipate a transition to or from a cognitive state of the user; and
provide, to the intelligent-facilitation subsystem, a signal indicating the transition to or from the cognitive state of the user.

16. The system of claim 15, wherein:
the one or more biosensors comprise one or more eye-tracking sensors adapted to measure gaze dynamics of the user;
the one or more biosignals comprise signals indicative of the gaze dynamics of the user; and
the gaze dynamics of the user are used to anticipate the transition to or from the cognitive state of the user.

17. The system of claim 15, wherein:
the one or more biosensors comprise one or more hand-tracking sensors;
the one or more biosignals comprise signals indicative of hand dynamics of the user; and
the signals indicative of hand dynamics of the user are used to anticipate the transition to or from the cognitive state of the user.

18. The system of claim 15, wherein:
the one or more biosensors comprise one or more neuromuscular sensors;
the one or more biosignals comprise neuromuscular signals obtained from the user's body; and
the neuromuscular signals obtained from the user's body are used to anticipate the transition to or from the cognitive state of the user.

19. The system of claim 15, wherein:
the system is an extended-reality system;
the intelligent-facilitation subsystem is further adapted to:
receive the signal indicating the transition to or from the cognitive state of the user; and
perform, in response to receiving the signal, the one or more assistive actions to reduce the user's cognitive load.

20. A non-transitory computer-readable medium comprising one or more computer-executable instructions that, when executed by at least one processor of a computing device, cause the computing device to:
acquire, via one or more biosensors, one or more biosignals generated by a user of the computing device, the computing system comprising an intelligent-facilitation subsystem adapted to perform one or more assistive actions to reduce the user's cognitive load;
use the one or more biosignals to anticipate a transition to or from a cognitive state of the user; and
provide, to the intelligent-facilitation subsystem, a signal indicating the transition to or from the cognitive state of the user.
